SEQUENCE LISTING (101 sequences)

SEQ ID NO: 1 - protein, 12 aa, Artificial Sequence
  chemically-synthesized D-peptide
TAWYANFEKLLR

SEQ ID NO: 2 - protein, 10 aa, Artificial Sequence
  chemically-synthesized D-peptide
WYANFEKLLR

SEQ ID NO: 3 - protein, 12 aa, Artificial Sequence
  chemically-synthesized D-peptide
TAXYANFEKLLR

SEQ ID NO: 4 - protein, 12 aa, Artificial Sequence
  chemically-synthesized D-peptide
TAXYANFEKLLR

SEQ ID NO: 5 - protein, 10 aa, Artificial Sequence
  chemically-synthesized D-peptide
XYANFEKLLR

SEQ ID NO: 6 - protein, 10 aa, Artificial Sequence
  chemically-synthesized D-peptide
XYANFEKLLR

SEQ ID NO: 7 - protein, 12 aa, Artificial Sequence
  chemically-synthesized D-peptide
TAWYANXEKLLR

SEQ ID NO: 8 - protein, 12 aa, Artificial Sequence
  chemically-synthesized D-peptide
TAWYANXEKLLR

SEQ ID NO: 9 - protein, 10 aa, Artificial Sequence
  chemically-synthesized D-peptide
WYANXEKLLR

SEQ ID NO: 10 - protein, 10 aa, Artificial Sequence
  chemically-synthesized D-peptide
WYANXEKLLR

SEQ ID NO: 11 - protein, 12 aa, Artificial Sequence
  chemically-synthesized D-peptide
TAWYANFEKLXR

SEQ ID NO: 12 - protein, 10 aa, Artificial Sequence
  chemically-synthesized D-peptide
WYANFEKLXR

SEQ ID NO: 13 - protein, 12 aa, Artificial Sequence
  chemically-synthesized D-peptide
TAXYANXEKLLR

SEQ ID NO: 14 - protein, 12 aa, Artificial Sequence
  chemically-synthesized D-peptide
TAXYANXEKLLR

SEQ ID NO: 15 - protein, 12 aa, Artificial Sequence
  chemically-synthesized D-peptide
TAXYANXEKLLR

SEQ ID NO: 16 - protein, 12 aa, Artificial Sequence
  chemically-synthesized D-peptide
TAXYANXEKLLR

SEQ ID NO: 17 - protein, 12 aa, Artificial Sequence
  chemically-synthesized D-peptide
TAXYANXEKLLR

SEQ ID NO: 18 - protein, 12 aa, Artificial Sequence
  chemically-synthesized D-peptide
TAXYANXEKLLR

SEQ ID NO: 19 - protein, 10 aa, Artificial Sequence
  chemically-synthesized D-peptide
XYANXEKLLR

SEQ ID NO: 20 - protein, 10 aa, Artificial Sequence
  chemically-synthesized D-peptide
XYANXEKLLR

SEQ ID NO: 21 - protein, 10 aa, Artificial Sequence
  chemically-synthesized D-peptide
XYANXEKLLR

SEQ ID NO: 22 - protein, 10 aa, Artificial Sequence
  chemically-synthesized D-peptide
XYANXEKLLR

SEQ ID NO: 23 - protein, 12 aa, Artificial Sequence
  chemically-synthesized D-peptide
TAWYANXEKLXR

SEQ ID NO: 24 - protein, 12 aa, Artificial Sequence
  chemically-synthesized D-peptide
TAWYANXEKLXR

SEQ ID NO: 25 - protein, 10 aa, Artificial Sequence
  chemically-synthesized D-peptide
WYANXEKLXR

SEQ ID NO: 26 - protein, 10 aa, Artificial Sequence
  chemically-synthesized D-peptide
WYANXEKLXR

SEQ ID NO: 27 - protein, 12 aa, Artificial Sequence
  chemically-synthesized D-peptide
TAXYANXEKLXR

SEQ ID NO: 28 - protein, 12 aa, Artificial Sequence
  chemically-synthesized D-peptide
TAXYANXEKLXR

SEQ ID NO: 29 - protein, 12 aa, Artificial Sequence
  chemically-synthesized D-peptide
TAXYANXEKLXR

SEQ ID NO: 30 - protein, 12 aa, Artificial Sequence
  chemically-synthesized D-peptide
TAXYANXEKLXR

SEQ ID NO: 31 - protein, 10 aa, Artificial Sequence
  chemically-synthesized D-peptide
XYANXEKLXR

SEQ ID NO: 32 - protein, 10 aa, Artificial Sequence
  chemically-synthesized D-peptide
XYANXEKLXR

SEQ ID NO: 33 - protein, 10 aa, Artificial Sequence
  chemically-synthesized D-peptide
XYANXEKLXR

SEQ ID NO: 34 - protein, 10 aa, Artificial Sequence
  chemically-synthesized D-peptide
XYANXEKLXR

SEQ ID NO: 35 - protein, 12 aa, Artificial Sequence
  chemically-synthesized D-peptide
TNWYANLEKLLR

SEQ ID NO: 36 - protein, 10 aa, Artificial Sequence
  chemically-synthesized D-peptide
WYANLEKLLR

SEQ ID NO: 37 - protein, 12 aa, Artificial Sequence
  chemically-synthesized D-peptide
TNXYANLEKLLR

SEQ ID NO: 38 - protein, 10 aa, Artificial Sequence
  chemically-synthesized D-peptide
XYANLEKLLR

SEQ ID NO: 39 - protein, 12 aa, Artificial Sequence
  chemically-synthesized D-peptide
TNWYANXEKLLR

SEQ ID NO: 40 - protein, 10 aa, Artificial Sequence
  chemically-synthesized D-peptide
WYANXEKLLR

SEQ ID NO: 41 - protein, 12 aa, Artificial Sequence
  chemically-synthesized D-peptide
TNWYANLEKLXR

SEQ ID NO: 42 - protein, 10 aa, Artificial Sequence
  chemically-synthesized D-peptide
WYANLEKLXR

SEQ ID NO: 43 - protein, 12 aa, Artificial Sequence
  chemically-synthesized D-peptide
TNXYANXEKLLR

SEQ ID NO: 44 - protein, 10 aa, Artificial Sequence
  chemically-synthesized D-peptide
XYANXEKLLR

SEQ ID NO: 45 - protein, 12 aa, Artificial Sequence
  chemically-synthesized D-peptide
TNWYANXEKLXR

SEQ ID NO: 46 - protein, 10 aa, Artificial Sequence
  chemically-synthesized D-peptide
WYANXEKLXR

SEQ ID NO: 47 - protein, 12 aa, Artificial Sequence
  chemically-synthesized D-peptide
TNXYANXEKLXR

SEQ ID NO: 48 - protein, 10 aa, Artificial Sequence
  chemically-synthesized D-peptide
XYANXEKLXR

SEQ ID NO: 49 - protein, 12 aa, Artificial Sequence
  chemically-synthesized D-peptide
DWWPLAFEALLR

SEQ ID NO: 50 - protein, 10 aa, Artificial Sequence
  chemically-synthesized D-peptide
WPLAFEALLR

SEQ ID NO: 51 - protein, 12 aa, Artificial Sequence
  chemically-synthesized D-peptide
DWXPLAFEALLR

SEQ ID NO: 52 - protein, 10 aa, Artificial Sequence
  chemically-synthesized D-peptide
XPLAFEALLR

SEQ ID NO: 53 - protein, 12 aa, Artificial Sequence
  chemically-synthesized D-peptide
DWWPLAXEALLR

SEQ ID NO: 54 - protein, 10 aa, Artificial Sequence
  chemically-synthesized D-peptide
WPLAXEALLR

SEQ ID NO: 55 - protein, 12 aa, Artificial Sequence
  chemically-synthesized D-peptide
DWWPLAFEALXR

SEQ ID NO: 56 - protein, 10 aa, Artificial Sequence
  chemically-synthesized D-peptide
WPLAFEALXR

SEQ ID NO: 57 - protein, 12 aa, Artificial Sequence
  chemically-synthesized D-peptide
DWXPLAXEALLR

SEQ ID NO: 58 - protein, 10 aa, Artificial Sequence
  chemically-synthesized D-peptide
XPLAXEALLR

SEQ ID NO: 59 - protein, 12 aa, Artificial Sequence
  chemically-synthesized D-peptide
DWWPLAXEALXR

SEQ ID NO: 60 - protein, 10 aa, Artificial Sequence
  chemically-synthesized D-peptide
WPLAXEALXR

SEQ ID NO: 61 - protein, 12 aa, Artificial Sequence
  chemically-synthesized D-peptide
DWXPLAXEALXR

SEQ ID NO: 62 - protein, 10 aa, Artificial Sequence
  chemically-synthesized D-peptide
XPLAXEALXR

SEQ ID NO: 63 - protein, 12 aa, Artificial Sequence
  chemically-synthesized D-peptide
TSFAEYWNLLSP

SEQ ID NO: 64 - protein, 10 aa, Artificial Sequence
  chemically-synthesized D-peptide
WYANFEKLLA

SEQ ID NO: 65 - protein, 12 aa, Artificial Sequence
  chemically-synthesized D-peptide
TAWYANFEKLLA

SEQ ID NO: 66 - protein, 10 aa, Artificial Sequence
  chemically-synthesized D-peptide
WYANFEALLR

SEQ ID NO: 67 - protein, 12 aa, Artificial Sequence
  chemically-synthesized D-peptide
TAWYANFEALLR

SEQ ID NO: 68 - protein, 10 aa, Artificial Sequence
  chemically-synthesized D-peptide
WYANFAKLLA

SEQ ID NO: 69 - protein, 12 aa, Artificial Sequence
  chemically-synthesized D-peptide
TAWYANFAKLLA

SEQ ID NO: 70 - protein, 10 aa, Artificial Sequence
  chemically-synthesized D-peptide
WYXNFEKLLX

SEQ ID NO: 71 - protein, 12 aa, Artificial Sequence
  chemically-synthesized D-peptide
TAWYXNFEKLLX

SEQ ID NO: 72 - protein, 10 aa, Artificial Sequence
  chemically-synthesized D-peptide
WYXNFEXLLR

SEQ ID NO: 73 - protein, 12 aa, Artificial Sequence
  chemically-synthesized D-peptide
TAWYXNFEXLLR

SEQ ID NO: 74 - protein, 12 aa, Artificial Sequence
  chemically-synthesized D-peptide
DWWPXAFEXLLR

SEQ ID NO: 75 - protein, 12 aa, Artificial Sequence
  chemically-synthesized D-peptide
DWWPXAFEALLX

SEQ ID NO: 76 - protein, 12 aa, Artificial Sequence
  chemically-synthesized D-peptide
DWWPLXFEXLLR

SEQ ID NO: 77 - protein, 12 aa, Artificial Sequence
  chemically-synthesized D-peptide
DWWPLXFEALLX

SEQ ID NO: 78 - protein, 12 aa, Artificial Sequence
  chemically-synthesized D-peptide
DWWPLAFXALLX

SEQ ID NO: 79 - protein, 12 aa, Artificial Sequence
  chemically-synthesized D-peptide
DWWPLAFEXLLX

SEQ ID NO: 80 - protein, 12 aa, Artificial Sequence
  chemically-synthesized D-peptide
TAWYXNFEXLLR

SEQ ID NO: 81 - protein, 10 aa, Artificial Sequence
  chemically-synthesized D-peptide
WYXNFEXLLR

SEQ ID NO: 82 - protein, 12 aa, Artificial Sequence
  chemically-synthesized D-peptide
TAWYXNFEKLLX

SEQ ID NO: 83 - protein, 10 aa, Artificial Sequence
  chemically-synthesized D-peptide
WYXNFEKLLX

SEQ ID NO: 84 - protein, 12 aa, Artificial Sequence
  chemically-synthesized D-peptide
TAWYAXFEXLLR

SEQ ID NO: 85 - protein, 10 aa, Artificial Sequence
  chemically-synthesized D-peptide
WYAXFEXLLR

SEQ ID NO: 86 - protein, 12 aa, Artificial Sequence
  chemically-synthesized D-peptide
TAWYAXFEKLLX

SEQ ID NO: 87 - protein, 10 aa, Artificial Sequence
  chemically-synthesized D-peptide
WYAXFEKLLX

SEQ ID NO: 88 - protein, 12 aa, Artificial Sequence
  chemically-synthesized D-peptide
TAWYANFXKLLX

SEQ ID NO: 89 - protein, 10 aa, Artificial Sequence
  chemically-synthesized D-peptide
WYANFXKLLX

SEQ ID NO: 90 - protein, 12 aa, Artificial Sequence
  chemically-synthesized D-peptide
TAWYANFEXLLX

SEQ ID NO: 91 - protein, 10 aa, Artificial Sequence
  chemically-synthesized D-peptide
WYANFEXLLX

SEQ ID NO: 92 - protein, 12 aa, Artificial Sequence
  chemically-synthesized D-peptide
DWXPLAFEALXR

SEQ ID NO: 93 - protein, 10 aa, Artificial Sequence
  chemically-synthesized D-peptide
XPLAFEALXR

SEQ ID NO: 94 - protein, 12 aa, Artificial Sequence
  chemically-synthesized D-peptide
DXWPLAFEALLR

SEQ ID NO: 95 - protein, 12 aa, Artificial Sequence
  chemically-synthesized D-peptide
DXWPLAXEALLR

SEQ ID NO: 96 - protein, 12 aa, Artificial Sequence
  chemically-synthesized D-peptide
DXWPLAFEALXR

SEQ ID NO: 97 - protein, 12 aa, Artificial Sequence
  chemically-synthesized D-peptide
DXWPLAXEALXR

SEQ ID NO: 98 - protein, 12 aa, Artificial Sequence
  chemically-synthesized D-peptide
DXXPLAFEALLR

SEQ ID NO: 99 - protein, 12 aa, Artificial Sequence
  chemically-synthesized D-peptide
DXXPLAXEALLR

SEQ ID NO: 100 - protein, 12 aa, Artificial Sequence
  chemically-synthesized D-peptide
DXXPLAFEALXR

SEQ ID NO: 101 - protein, 12 aa, Artificial Sequence
  chemically-synthesized D-peptide
DXXPLAXEALXR

(19) United States
(12) Patent Application Publication
(10) Pub. No.: US 2012/0328692 A1
(43) Pub. Date: Dec. 27, 2012

(54) POTENT D-PEPTIDE ANTAGONISTS OF MDM2 AND MDMX FOR ANTICANCER THERAPY

(75) Inventors: Wuyuan LU, Ellicott City, MD (US); Changyou ZHAN, Catonsville, MD (US)

(73) Assignee: UNIVERSITY OF MARYLAND, BALTIMORE, Baltimore, MD (US)

(21) Appl. No.: 13/523,532

(22) Filed: Jun. 14, 2012

Related U.S. Application Data
(60) Provisional application No. 61/500,674, filed on Jun. 24, 2011.

(51) Int. Cl. A61K 38/10; A61K 38/08; A61P 35/00; C07K 7/06; C07K 7/08; A61K 9/127

(52) U.S. Cl. 424/450; 530/327; 514/19.3; 530/328

(57) ABSTRACT

The present invention relates to a group of MDM2 and MDMX antagonists, namely, D-peptides, variants thereof, and stapled D-peptides, along with pharmaceutical compositions comprising the antagonists, and methods of treating conditions such as cancer using the antagonists.

### CROSS-REFERENCE TO RELATED APPLICATIONS

The present application claims priority to U.S. provisional application No. 61/500,674, filed Jun. 24, 2011, the entire disclosure of which is herein incorporated by reference in its entirety.

### BACKGROUND OF THE INVENTION

1. Field of the Invention

The present invention relates to D-peptides and hydrocarbon-stapled versions thereof that are antagonists of MDM2 and MDMX and that inhibit p53-MDM2/MDMX interactions. The invention is further related to pharmaceutical compositions comprising the antagonists, and methods of treating conditions such as cancer using the antagonists.

2. Background of the Invention

p53 is a tumor suppressor that transcriptionally regulates, in response to cellular stresses such as DNA damage or oncogene activation, the expression of various target genes that mediate cell-cycle arrest, DNA repair, senescence or apoptosis (Levine A J and Oren M. Nat Rev Cancer 9(10):749-58 (2009); Vogelstein B, et al. Nature 408(6810):307-10 (2000)). Loss of p53 activity—either by somatic mutation of the TP53 gene or by functional inhibition of the p53 protein—is a common feature of human tumors. It is estimated that 50% of human tumors carry loss-of-function mutations in TP53, many of which are associated with malignant progression, poor prognosis and resistance to treatment (Hainaut P and Hollstein M. Adv Cancer Res 77:81-137 (2000); Soussi T and Wiman K G. Cancer Cell 12(4):303-12 (2007)).

In other tumors, however, p53 is present in its wild-type form. In these tumors displaying normal p53 function and levels, high levels of negative regulators of p53 such as the E3 ubiquitin ligase MDM2 and its homolog MDMX (also known as MDM4) impede p53-induced growth inhibitory and apoptotic responses (Brown C J, et al. Nat Rev Cancer 9(12):862-73 (2009); Levine A J. Cell 88(3):323-31 (1997); Wade M, et al. Trends Cell Biol 20(5):299-309 (2010)). MDM2 primarily controls p53 stability by targeting the tumor suppressor protein for ubiquitin-mediated constitutive degradation by the proteasome (Haupt Y, et al. Nature 387(6630):296-9 (1997)), whereas MDMX mainly functions as an effective transcriptional antagonist of p53 that blocks its ability to regulate responsive genes expression (Shvarts A, et al. Embo J 15(19):5349-57 (1996)). Gene amplification and over-expression of MDM2 and MDMX, found in a significant fraction of cancers without concomitant p53 mutation, correlate to p53 inactivation and tumor survival (Marine J C, et al. J Cell Sci 120(Pt 3):371-8 (2007); Toledo F and Wahl G M. Nat Rev Cancer 6(12):909-23 (2006)). Both in vitro and in vivo data demonstrate that inhibition of the p53-MDM2/MDMX interaction by MDM2/MDMX antagonists re-activates the p53 pathway, leading to selective killing of tumor cells in a p53-dependent manner (Laurie N A, et al. Nature 444(7115):61-6 (2006); Shangary S, et al. Proc Natl Acad Sci USA 105(10):3933-8 (2008); Vassilev L T, et al. Science 303(5659):844-8 (2004)). Acting synergistically in tumor cells to inactivate the p53 pathway, MDM2 and MDMX are among the most promising molecular targets for anticancer therapy.

There exist two major classes of antagonists that target the p53-binding domain of MDM2/MDMX, i.e., low molecular weight, non-peptidic compounds and peptide inhibitors (Brown C J, et al. Nat Rev Cancer 9(12):862-73 (2009); Murray J K and Gellman S H. Biopolymers 88(5):657-86 (2007); Shangary S and Wang S. Annu Rev Pharmacol Toxicol 49:223-41 (2009); Wade M and Wahl G M. Mol Cancer Res 7(1):1-11 (2009)). Small molecules, by virtue of their small size, low price, oral availability, and ability to cross membranes, are traditionally preferred drug candidates, but suffer from drawbacks including rapid clearance and metabolism. Peptides, on the other hand, can be more potent, of higher specificity and of lower toxicity. Two major drawbacks of peptides, however, severely limit their therapeutic value. Peptides generally exhibit excessive backbone flexibility and poor membrane permeability, both of which can hinder their use as a practical alternative to small molecules. Conformational flexibility of a peptide not only sacrifices its binding affinity for target protein due to entropy loss, but also contributes to its proteolytic susceptibility or poor in vivo stability.

What is needed, therefore, is a practical alternative to small molecules and protease-susceptible peptides for the treatment of tumors that exhibit wild-type p53 through the antagonism of p53-MDM2/MDMX interactions.

### SUMMARY OF THE INVENTION

The present invention relates to D-peptides that bind with high affinity to MDM2 and/or MDMX, and that antagonize the ability of the ligases to regulate p53 activity in vivo. Certain embodiments of the invention related to the peptides are set forth in SEQ ID NOs:1, 2, 35, 36, 49 and 50.

The present invention relates to variants of the D-peptides that bind with increased affinity to MDM2 and/or MDMX, and that also antagonize the ability of the ligases to regulate p53 activity in vivo. The variants have altered side-groups on at least one amino acid of the D-peptide that imparts the increased affinity compared to the wild-type D-peptide. Certain embodiments of the invention related to the variants are set forth in SEQ ID NOs:3-34, 37-48, 51-62 and 92-101.

The present invention relates to hydrocarbon-stapled D-peptides that again bind with high affinity to MDM2 and/or MDMX, and that antagonize the ability of the ligases to regulate p53 activity in vivo. The stapled D-peptides are prepared using the D-peptides and variants of the D-peptides of the present invention. Certain embodiments of the invention related to the stapled D-peptides are set forth in Table 4A, 5A and 5B.

The present invention relates to pharmaceutical compositions comprising the D-peptides, variants, and stapled D-peptides.

The present invention relates to methods of treating cancer in a subject, such as a malignant tumor, comprising administering a pharmaceutical composition of the present invention to a subject having cancer.

In a first embodiment, the present invention provides a D-peptide that binds MDM2 or MDMX, or both, with high affinity and that is selected from the group consisting of SEQ ID NOs:1, 2, 49 and 50.

In a second embodiment, the present invention provides a variant of D-peptide that binds MDM2 or MDMX, or both, with high affinity and that is selected from the group consisting of SEQ ID NOs3-34, 37-48, and 51-62.

In a third embodiment, the present invention provides a stapled D-peptide that binds MDM2 or MDMX, or both, with high affinity selected from a stapled version of a D-peptide selected from the group consisting of SEQ ID NOs: 1, 3, 4, 7, 8, 11, 13-18, 23-24, 27-30, 35, 37, 39, 41, 43, 45, 47, 49, 51, 53, 55, 57, 59 and 61.

In a fourth embodiment, the present invention provides a stapled D-peptide that binds MDM2 or MDMX, or both, with high affinity selected from a stapled version of a variant of a D-peptide selected from the group consisting of SEQ ID NOs:2, 5, 6, 9, 10, 12, 19-22, 25, 26, 31-34, 36, 38, 40, 42, 44, 46, 48, 50, 52, 54, 56, 58, 60 and 62.

In a fifth embodiment, the present invention provides a stapled D-peptide that binds MDM2 or MDMX, or both, with high affinity and that is selected from the group consisting of SEQ ID NOs:64-73.

In a sixth embodiment, the present invention provides a stapled D-peptide that binds MDM2 or MDMX, or both, with high affinity and that is selected from the group consisting of SEQ ID NOs:74-79.

In a seventh embodiment, the present invention provides a stapled D-peptide that binds MDM2 or MDMX, or both, with high affinity and that is selected from the group consisting of SEQ ID NOs:80-91.

In an eighth embodiment, the present invention provides (i) polynucleotides encoding the D-peptides of the present invention, (ii) polynucleotides encoding the variants of a D-peptide of the present invention, and (iii) polynucleotides encoding the stapled D-peptides of the present invention.

In a ninth embodiment, the present invention provides vectors comprising one or more of the polynucleotides of the present invention.

In a tenth embodiment, the present invention provides host cells comprising one or more of the vectors of the present invention. In a related embodiment, the present invention provides methods of producing one or more of the D-peptides of the present invention, and/or one or more of the variants of a D-peptide, and/or one or more of the stapled D-peptides, comprising culturing one or more of the host cells of the present invention under conditions promoting expression of the polynucleotide encoding a D-peptide, a variant of a D-peptide or a stapled D-peptide.

In an eleventh embodiment, the present invention provides pharmaceutical compositions comprising (i) one or more of the D-peptides of the present invention, and/or (ii) one or more of the variants of a D-peptide, and/or (iii) one or more of the stapled D-peptides of the present invention, and a pharmaceutical carrier and/or excipient.

In a twelfth embodiment, the present invention provides methods of treating a subject having cancer, comprising administering to a subject having cancer a pharmaceutically-effective amount of one or more of the pharmaceutical compositions of the present invention, thereby treating a subject having cancer. In one aspect of this embodiment, the subject is a human. In some aspects, the carrier is a PEGylated liposome. In some aspects, the carrier is a PEGylated liposome coated via a PEG spacer with a cyclic RGD peptide c(RGD^{D}YK).

In a thirteenth embodiment, the present invention provides compositions comprising (i) one or more of the D-peptides, and/or (ii) one or more of the variants of a D-peptide, and/or (iii) one or more of the stapled D-peptides, and a carrier and/or excipient. In some aspects, the carrier is a PEGylated liposome. In some aspects, the carrier is a PEGylated liposome is coated via a PEG spacer with a cyclic RGD peptide c(RGD^{D}YK).

### BRIEF DESCRIPTION OF THE DRAWINGS

FIG. 1 depicts the crystal structures of phage-selected ^{D}PMI-α (left; SEQ ID NO:35) and ^{D}PMI-γ (right; SEQ ID NO:49) bound to synthetic MDM2. Critical residues involved in molecular recognition are shown in stick.

FIG. 2 depicts (top) growth inhibition of human glioblastoma U87 cells (3,000 cells/well) by Nutlin-3, free ^{D}PMI-α, liposome-^{D}PMI-α, RGD-liposome-^{D}PMI-α, and RGD-liposome-^{L}PMI-α as determined by the standard MTS cell viability assay after a three-day treatment. The inhibition curves are averages of 3 independent measurements. (Bottom) Western blot analysis of p53, MDM2, p21, and beta-actin expression in U87 and U251 cells 12 h after treatment with indicated concentrations of RGD-liposome-^{D}PMI-α (10% SDS-PAGE).

FIG. 3 depicts binding of ^{D}PMI-β and 4-CF₃-Phe7-^{D}PMI-β for MDM2 quantified by competition SPR is shown to the left, and the co-crystal structure of 4-CF₃-Phe7-^{D}PMI-β and MDM2 shown to the right.

FIG. 4 depicts the structure of MDM2 bound ^{D}PMI-α. Trp3, Leu7, and Leu11 constitute the MDM2-binding interface (left). Some commercially available non-classical amino acids bearing an olefinic side chain in both R and S forms (right).

FIG. 5 depicts the co-crystal structure of ^{D}PMI-alpha and MDM2, demonstrating that position 7 of the indole ring of Trp3 points directly towards a pocket, which is only partially filled. Therefore, introduction of additional chemical groups at position 7 may significantly improve binding affinity.

### DETAILED DESCRIPTION OF THE INVENTION

The p53 protein, encoded by the TP53 gene, is a well-known protein in the art and it is known to regulate the expression of various target genes that are associated with cell-cycle arrest. In general, expression and activity of p53 results in either cell-cycle arrest, DNA repair, senescence and/or apoptosis of the cell. Consistent with this activity, the loss of p53, either in expression or in activity, is a common feature in tumor cells, where cell growth and division are unchecked. It is estimated that 50% of human tumors carry loss-of-function mutations in TP53, many of which are associated with malignant progression, poor prognosis and resistance to treatment.

There are well-known methods for assaying p53 activity in cells. For example, cell viability assays can be used to determine the activity of p53 in treated and untreated cells. Other methods for determining p53 activity include, but are not limited to, monitoring levels of proteins whose expression is controlled by p53, the use of commercially available reporter assays that monitor p53 activity, quantifying p53 levels, monitoring p53-dependent apoptosis or growth arrest, monitoring tumor growth, etc. Another method of indirectly determining p53 activity includes monitoring the activity or presence of active MDM2 and/or MDMX proteins.

MDMX and MDM2 are non-redundant inhibitors of p53, as both MDMX and MDM2 are often required to inhibit p53 activity in the same cell type and each inhibitor is unable to compensate for the loss of the other. It appears that, in unstressed cells, MDM2 primarily controls p53 stability (levels) through ubiquitination, whereas MDMX mainly functions as a significant p53 transcriptional antagonist, independent of MDM2 activity. Under stress conditions, it appears that MDM2 and MDMX cooperate to activate p53 through mechanisms involving both MDM2 auto-degradation (auto-ubiquitination) and MDM2-dependent degradation of MDMX.

MDM2 (murine double minute 2 protein) is an E3 ubiquitin ligase that primarily controls p53 stability by targeting it for ubiquitin-mediated constitutive degradation by the proteasome. MDMX, which is a homolog of MDM2 and is also known as MDM4, mainly functions as an effective transcriptional antagonist of p53 that blocks its ability to regulate responsive gene expression. Thus, in many tumors where p53 is present in its wild-type form, high levels of these negative regulators impede p53-induced growth inhibition and apoptosis. Indeed, gene amplification and over-expression of MDM2 and MDMX are found in a significant fraction of cancers, without concomitant p53 mutation, correlating highly with tumor survival.

The human form of MDM2 is 491 amino acids and comprises an N-terminal p53 binding domain, a central acidic domain, preceded by nuclear export and localization signals essential for nuclear-cytoplasmic trafficking of MDM2, a Zinc finger domain, and a C-terminal Zinc-dependent RING finger domain that confers E3 ubiquitin ligase activity. MDM2 appears to negatively regulate p53 activity through three distinctive mechanisms involving the regulation of protein activity, in vivo stability and subcellular localization. First, MDM2 binds to the p53 transactivation domain, thereby inhibiting p53-mediated transactivation. Second, MDM2 ubiquitylates p53 to target the tumor suppressor protein for constitutive degradation by the proteasome. Third, binding of MDM2 triggers transport of p53 from the nucleus to the cytoplasm (Momand J, et al. Cell 69(7):1237-45 (1992); Oliner J D, et al. Nature 362(6423):857-60 (1993); Wallace M, et al. Mol Cell 23(2):251-63 (2006); Yu G W, et al. Proc Natl Acad Sci USA 103(5):1227-32 (2006); Deshaies R J and Joazeiro C A. Annu Rev Biochem 78:399-434 (2009); Haupt Y, et al. Nature 387(6630):296-9 (1997); Honda R, et al. FEBS Lett 420(1):25-7 (1997); Kubbutat M H, et al. Nature 387(6630):299-303 (1997)). Mdm2 knockout mice die extremely early during development due to increased apoptosis that is mediated by uncontrolled p53 activity (Jones S N, et al. Nature 378(6553):206-8 (1995); Montes de Oca Luna R, et al. Nature 378(6553):203-6 (1995); Parant J, et al. Nat Genet. 29(1):92-5 (2001)). A double knockout of both Mdm2 and TP53, however, rescues the early embryonic lethality, demonstrating the importance of MDM2 in the control of p53 activity. The amino acid sequence of human MDM2 is located at GenBank Accession No. Q00987, the entire record of which is incorporated by reference. GenBank can be accessed via the world wide web at ncbi.nlm.nih.gov.

MDMX (also known as MDM4) was first discovered as a p53-binding protein in cells. Structurally related to MDM2, human MDMX consists of 490 amino acids and possesses domain structures arranged similarly to MDM2 (Shvarts A, et al. Embo J 15(19):5349-57 (1996)). In particular, the N-terminal p53-binding domains of MDM2 and MDMX are highly homologous with an over 50% sequence identity. Expectedly, MDMX also inhibits p53 transactivation. Unlike MDM2, however, MDMX lacks ubiquitin-ligase function and is not transcriptionally activated by p53 in response to DNA damage. Nevertheless, deletion of the Mdmx gene in mice also causes early embryonic lethality that is rescued by p53 inactivation. The amino acid sequence of human MDM2 is located at GenBank Accession No. 015151, the entire record of which is incorporated by reference.

The present invention relates to D-peptides, variants thereof, and hydrocarbon-stapled D-peptides prepared from the D-peptides and variants, each of which binds with high affinity to MDM2 and/or MDMX. The D-peptides, variants, and stapled D-peptides of the present invention have the further ability of antagonize the MDM2 and/or MDMX regulation of p53 activity in vivo. Collectively, and as used herein, the D-peptides, variants, and stapled D-peptides of the present invention are referred to as “the MDM2/MDMX antagonists” of the present invention.

D-Peptides

The present invention relates to D-peptides that bind with high affinity to MDM2 and/or MDMX, and that antagonize the ability of the ligases to regulate p53 activity in vivo. As used herein, the term “D-peptide” has the ordinary and customary meaning of the term, namely, a peptide comprising D-amino acids linked by amide bonds. The D-peptides of the present invention are generally comprised of 10 or 12 D-amino acids. Tables 1-3 provides examples of D-peptides of the present invention, which include the D-peptides of SEQ ID NOs:1, 2, 35, 36, 49 and 50.

### Variants

The present invention also relates to variants of the D-peptides, where one or more amino acids has an altered side-group R, of the generic formula H₂NCHRCOOH for amino acids. The variants have an increased affinity for MDM2 and/or MDMX compared with the “wild-type” D-peptide on which they are based, and thus also bind to MDM2 and/or MDMX with high affinity. And as with the D-peptides, the variants antagonize the ability of the ligases to regulate p53 activity in vivo.

In particular, in 1, 2, 3, 4, 5, 6, 7, 8, 9, 10, 11 or 12 of the amino acids in the 12 amino acid D-peptides, and in 1, 2, 3, 4, 5, 6, 7, 8, 9 or 10 of the amino acids in the 10 amino acid D-peptides, the naturally-occurring side-groups R are independently modified by F, Cl, Br, I, CH₃, CF₃, CN, OH, or NO₂. In particular embodiments, 1, 2 or 3 of the amino acids in the D-peptides have the naturally-occurring side-group R independently modified by F, Cl, Br, I, CH₃, CF₃, CN, OH, or NO₂. Examples of such variants of D-peptides include, but are not limited to, SEQ ID NOs: 3-34, 37-48, 51-62 and 92-101 set forth in Tables 1-3.

The specific amino acid(s) having a side-group R modified by F, Cl, Br, I, CH₃, CF₃, CN, OH, or NO₂ will vary for each D-peptide depending on the particular amino acids that interact when the D-peptide binds MDM2 and/or MDMX. For example, in embodiments where the variants are based on the D-peptide ^{D}PMI-β (SEQ ID NO:3-34), the side-group of one or more of Trp3, Phe7, and Leu11 may be modified. In embodiments where the variants are based on the D-peptide ^{D}PMI-α (SEQ ID NO:37-48), the side-group of one or more of Trp3, Leu7, and Leu11 may be modified. In embodiments where the variants are based on the D-peptide ^{D}PMI-γ (SEQ ID NO:51-62 and 92-101), the side-group of one or more of Trp2, Trp3, Phe7, and Leu11 may be modified.

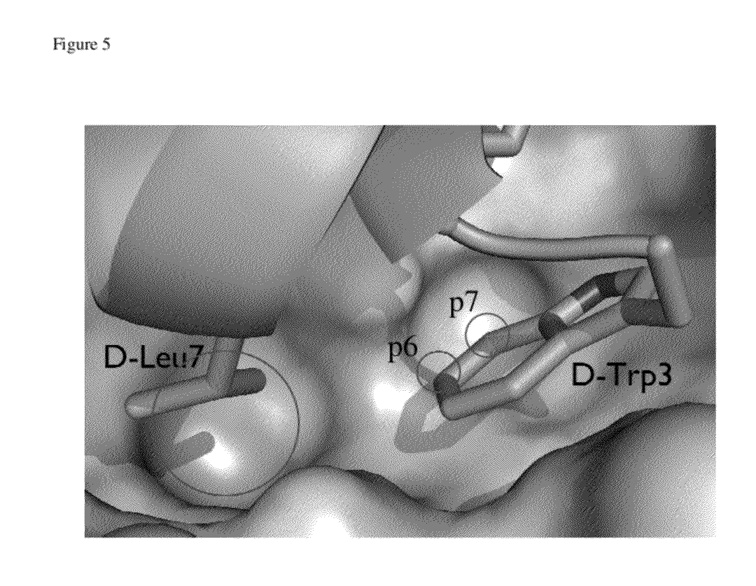

Further, the specific location on the side-group in which the modification will take place depends on the particular conformation between the D-peptide and MDM2 and/or MDMX. As shown in FIG. 5, both positions 6 and 7 of Trp3 interact with the binding pocket when ^{D}PMI-α binds MDM2. Thus, either of the two locations, or both of the locations may be modified by F, Cl, Br, I, CH₃, CF₃, CN, OH, or NO₂. In embodiments where the variants are based on the D-peptide ^{D}PMI-β (SEQ ID NO:3-34), the side-group at position 6 or 7, or both, of Trp3 may be modified; position 4 of Phe7 may be modified; positions γ, δ1 or δ2 on Leu11 may be modified. In embodiments where the variants are based on the D-peptide ^{D}PMI-α (SEQ ID NO:37-48), the side-group at position 6 or 7, or both, of Trp3 may be modified; positions γ, δ1 or δ2 of Leu7 may be modified; positions y, δ1 or δ2 on Leu11 may be modified. In embodiments where the variants are based on the D-peptide ^{D}PMI-γ (SEQ ID NO:51-62 and 92-101), the side-group at position 6 or 7, or both, of Trp3 may be modified; position 4 of Phe7 may be modified; positions γ, δ1 or δ2 on Leu11 may be modified.

### Stapled D-Peptides

Verdine and colleagues developed a hydrocarbon stapling technique that enables (i, i+3), (i, i+4) or (i, i+7) side-chain cross-linked and conformationally stabilized α-helical peptides to actively traverse the cell membrane with improved proteolytic stability and enhanced biological activity (Schafineister C E, et al. J Am Chem Soc 122:5891-92 (2000)). Hydrocarbon stapling approaches have been successfully applied to several different systems including a p53-derived peptide (Schafineister C E, et al. J Am Chem Soc 122:5891-92 (2000); Bernal F, et al. J Am Chem Soc 129(9):2456-7 (2007); Moellering R E, et al. Nature 462(7270):182-8 (2009); Walensky L D, et al. Science 305(5689):1466-70 (2004); Bautista A D, et al. J Am Chem Soc 132(9):2904-6 (2010)). The stapled p53 peptide, termed SAH-p53-8, bound MDM2 at an affinity of 55 nM as determined by fluorescence polarization, and induced p53-dependent apoptosis in SJSA-1 cells over-expressing MDM2 (Bernal F, et al. J Am Chem Soc 129(9):2456-7 (2007)). More recently, Bernal et al. reported that SAH-p53-8 bound MDMX with a K_{D} value of 2.3 nM—a 25-fold greater binding preference for MDMX over MDM2 (Cancer Cell 18(5):411-22 (2011)). The SAH-p53-8 peptide effectively induced p53-dependent killing of tumor cells by targeting MDM2, MDMX, or both, and significantly suppressed tumor growth in experimental animals bearing JEG-3 xenografts—an MDMX-expressing and Nutlin-3-resistant cancer (Bernal F, et al. Cancer Cell 18(5):411-22 (2011)).

The present invention encompasses hydrocarbon-stapled versions of the D-peptides and variants set forth in SEQ ID NOs:1-62 and 92-101. The hydrocarbon-stapled D-peptides of the present invention thus have a core comprised of D-amino acids linked by amide bonds, but with two of the amino acids substituted for by non-classical amino acids. The stapled D-peptides also bind with high affinity to MDM2 and/or MDMX, and antagonize the ability of the ligases to regulate p53 activity in vivo. The stapled D-peptides of the present invention include the 12 amino acid D-peptides of SEQ ID NOs:1, 3, 4, 7, 8, 11, 13-18, 23-24, 27-30, 35, 37, 39, 41, 43, 45, 47, 49, 51, 53, 55, 57, 59, 61, 92, and 94-101 having non-classical amino acids at positions 5+9, 5+12, 6+9, 6+12, 8+12 or 9+12. The stapled D-peptides of the present invention also include the 10 amino acid D-peptides of SEQ ID NOs:2, 5, 6, 9, 10, 12, 19-22, 25, 26, 31-34, 36, 38, 40, 42, 44, 46, 48, 50, 52, 54, 56, 58, 60, 62, and 93 having non-classical amino acids at positions 3+7, 3+10, 4+7, 4+10, 6+10 or 7+10.

The non-classical amino acids that may be used in the production of the stapled D-peptides of the present invention are any that contains an olefinic side chain. Suitable non-classical amino acids include, but are not limited to, (S)-2-(7′-octenyl)alanine, (R)-2-(7′-octenyl)alanine, (S)-2-(4′-pentenyl)alanine, (R)-2-(4′-pentenyl)alanine, and D-ornithine, shown forming the stapled D-peptides of Table 4 (SEQ ID NOs:64-73), and amino acids with olefinic side chains such as those forming the stapled D-peptides of Table 5 (SEQ ID NOs:74-91).

As mentioned above, the D-peptides, variants, and stapled D-peptides of the present invention are collectively referred to as “MDM2/MDMX antagonists” herein.

### Other Variations

The present invention also encompasses MDM2/MDMX antagonists having alternations in one or more amino acids, selected from deletions, additions and substitutions. For example, the MDM2/MDMX antagonists may comprise 1, 2, 3, 4, 5, 6, 7, 8, 9, 10 or 11 amino acid substitutions from the amino acid sequences of SEQ ID NOs:1, 3, 4, 7, 8, 11, 13-18, 23-24, 27-30, 35, 37, 39, 41, 43, 45, 47, 49, 51, 53, 55, 57, 59, 61, 92, and 94-101, or 1, 2, 3, 4, 5, 6, 7, 8, or 9 amino acid substitutions from the amino acid sequences of SEQ ID NOs:2, 5, 6, 9, 10, 12, 19-22, 25, 26, 31-34, 36, 38, 40, 42, 44, 46, 48, 50, 52, 54, 56, 58, 60, 62, and 93. Each amino acid substitution may independently be: (i) a change in the enantiomeric configuration of the amino acid (i.e., D or L), (ii) an amino acid substitution, such as a conservative amino acid substitution or a non-conservative amino acid substitution, where the substituted amino acid has the same enantiomeric configuration, or (iii) an amino acid substitution, such as a conservative amino acid substitution or a non-conservative amino acid substitution, where the substituted amino acid has a different enantiomeric configuration. Each of the substituted MDM2/MDMX antagonists retains high affinity for MDM2, MDMX, or both.

In an embodiment, amino acids at positions 3, 7 and 11 in SEQ ID NOs:1, 3, 4, 7, 8, 11, 13-18, 23-24, 27-30, 35, 37, 39, 41, 43, 45, 47, 49, 51, 53, 55, 57, 59, 61, 92, and 94-101 remain D-amino acids and 1, 2, 3, 4, 5, 6, 7, 8 or 9 of the amino acids at positions 1, 2, 4-6, 8-10 and 12 are L-amino acids. In another embodiment, amino acids at positions 1, 5 and 9 in SEQ ID NOs:2, 5, 6, 9, 10, 12, 19-22, 25, 26, 31-34, 36, 38, 40, 42, 44, 46, 48, 50, 52, 54, 56, 58, 60, 62, and 93 remain D-amino acids and 1, 2, 3, 4, 5, 6 or 7 of the amino acids at positions 2-4, 6-8 and 10 are L-amino acids.

The MDM2/MDMX antagonists may further comprise 1, 2, 3, 4, 5, 6, 7, 8, 9, 10 or 11 amino acid deletions from the amino acid sequences of SEQ ID NOs:1, 3, 4, 7, 8, 11, 13-18, 23-24, 27-30, 35, 37, 39, 41, 43, 45, 47, 49, 51, 53, 55, 57, 59, 61, 92, and 94-101, or 1, 2, 3, 4, 5, 6, 7, 8, or 9 amino acid deletions from the amino acid sequences of SEQ ID NOs:2, 5, 6, 9, 10, 12, 19-22, 25, 26, 31-34, 36, 38, 40, 42, 44, 46, 48, 50, 52, 54, 56, 58, 60, 62, and 93. The deletions may be deletions of one or more consecutive amino acids from the amino terminus or the carboxy terminus of the peptide, or one or more consecutive amino acids from within the peptide. As has been shown for some antagonists of SEQ ID NOs:2, 5, 6, 9, 10, 12, 19-22, 25, 26, 31-34, 36, 38, 40, 42, 44, 46, 48, 50, 52, 54, 56, 58, 60, 62, and 93, one or two amino acids may be deleted from the amino terminus without a significant decrease in measured affinity. Each of the deleted MDM2/MDMX antagonists retains high affinity for MDM2, MDMX, or both.

The MDM2/MDMX antagonists may also comprise 1, 2, 3, 4, 5, 6, 7, 8, 9 or 10, or more, amino acid additions to the amino acid sequences of SEQ ID NOs:1-62 and 92-101. Each amino acid addition may have the same or different enantiomeric configuration (i.e., D or L) in comparison to the majority configuration of the peptide. Each of the addition MDM2/MDMX antagonists retains high affinity for MDM2, MDMX, or both.

The skilled artisan will appreciate that a combination of deletions, additions and substitutions may be made to the MDM2/MDMX antagonists of SEQ ID NOs:1-62 and 92-101 as long as the resulting molecules retain high affinity for MDM2, MDMX, or both.

TABLE 1
^{D}PMI-β based D-Peptide and Variant Antagonists of MDM2 and MDMX

| | SEQ | |
|---|---|---|
| Name | ID NO: | Amino Acid Sequence |
| ^{D}PMI-β | 1 | TAWYANFEKLLR |
| ^{D}PMI-β-del | 2 | WYANFEKLLR |
| ^{D}PMI-β-W₁ | 3 | TAW₁YANFEKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-β-W₁(F) | 4 | TAW₁YANFEKLLR |
| | | W₁ = Trp with F at position 6 or position 7, or both positions |
| | | 6 and 7 |
| ^{D}PMI-β-W₁-del | 5 | W₁YANFEKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-β-W₁(F)-del | 6 | W₁YANFEKLLR |
| | | W₁ = Trp with F at position 6 or position 7, or both positions |
| | | 6 and 7 |
| ^{D}PMI-β-F₁ | 7 | TAWYANF₁EKLLR |
| | | F₁ = Phe with F, Cl, Br, I, CH₃, CF₃, CN, OH, or NO₂ at |
| | | position 4 |
| ^{D}PMI-β-F₁(CF₃) | 8 | TAWYANF₁EKLLR |
| | | F₁ = Phe with CF₃ at position 4 |
| ^{D}PMI-β-F₁-del | 9 | WYANF₁EKLLR |
| | | F₁ = Phe with F, Cl, Br, I, CH₃, CF₃, CN, OH, or NO₂ at |
| | | position 4 |
| ^{D}PMI-β-F₁(CF₃)-del | 10 | WYANF₁EKLLR |
| | | F₁ = Phe with CF₃ at position 4 |
| ^{D}PMI-β-L₁ | 11 | TAWYANFEKLL₁R |
| | | L₁ = Leu with F, Cl, Br, I, CH₃, CF₃, CN, OH, or NO₂ at |
| | | positions γ, δ1 or δ2 |
| ^{D}PMI-β-L₁-del | 12 | WYANFEKLL₁R |
| | | L₁ = Leu with F, Cl, Br, I, CH₃, CF₃, CN, OH, or NO₂ at |
| | | positions γ, δ1 or δ2 |
| ^{D}PMI-δ-W₁F₁ | 13 | TAW₁YANF₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-δ-W₁(F)F₁ | 14 | TAW₁YANF₁EKLLR |
| | | W₁ = Trp with F at position 6 or position 7, or both positions |
| | | 6 and 7 |
| | | F₁ = Phe with F, Cl, Br, I, CH₃, CF₃, CN, OH, or NO₂ at |
| | | position 4 |
| ^{D}PMI-δ-W₁F₁(CF₃) | 15 | TAW₁YANF₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with CF₃ at position 4 |
| ^{D}PMI-δ | 16 | TAW₁YANF₁EKLLR |
| | | W₁ = Trp with F at position 6 |
| | | F₁ = Phe with CF₃ at position 4 |
| ^{D}PMI-δ-F7 | 17 | TAW₁YANF₁EKLLR |
| | | W₁ = Trp with F at position 7 |
| | | F₁ = Phe with CF₃ at position 4 |
| ^{D}PMI-δ-F6, 7 | 18 | TAW₁YANF₁EKLLR |
| | | W₁ = Trp with F at positions 6 and 7 |
| | | F₁ = Phe With CF₃ at position 4 |
| ^{D}PMI-δ-W₁F₁-del | 19 | W₁YANF₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-δ-W₁(F)F₁-del | 20 | W₁YANF₁EKLLR |
| | | W₁ = Trp with F at position 6 or position 7, or both positions |
| | | 6 and 7 |
| | | F₁ = Phe with F, Cl, Br, I, CH₃, CF₃, CN, OH, or NO₂ at |
| | | position 4 |
| ^{D}PMI-δ-W₁F₁(CF₃)-del | 21 | W₁YANF₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with CF₃ at position 4 |
| ^{D}PMI-δ-W₁(F)F₁(CF₃)-del | 22 | W₁YANF₁EKLLR |
| | | W₁ = Trp with F at position 6 or position 7, or both positions |
| | | 6 and 7 |
| | | F₁ = Phe with CF₃ at position 4 |
| ^{D}PMI-δ-F₁L₁ | 23 | TAWYANF₁EKLLIR |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-F₁(CF₃)L₁ | 24 | TAWYANF₁EKLL₁R |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |
| ^{D}PMI-δ-F₁L₁-del | 25 | WYANF₁EKLL₁R |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-F₁(CF₃)L₁-del | 26 | WYANF₁EKLL₁R |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |
| ^{D}PMI-δ-W₁F₁L₁ | 27 | TAW₁YANF₁EKLL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-W₁(F)F₁L₁ | 28 | TAW₁YANF₁EKLL₁R |
| | | W₁ = Trp with F at position 6 or position 7, or both positions |
| | | 6 and 7 |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-W₁F₁(CF₃)L₁ | 29 | TAW₁YANF₁EKLL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |
| ^{D}PMI-δ-W₁(F)F₁(CF₃)L₁ | 30 | TAW₁YANF₁EKLL₁R |
| | | W₁ = Trp with F at position 6 or position 7, or both positions |
| | | 6 and 7 |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |
| ^{D}PMI-δ-W₁F₁L₁-del | 31 | W₁YANF₁EKLL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-W₁(F)F₁L₁-del | 32 | W₁YANF₁EKLL₁R |
| | | W₁ = Trp with F at position 6 or position 7, or both positions |
| | | 6 and 7 |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-δ-W₁F₁(CF₃)L₁-del | 33 | W₁YANF₁EKLL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |
| ^{D}PMI-δ-W₁(F)F₁(CF₃)L₁-del | 34 | W₁YANF₁EKLL₁R |
| | | W₁ = Trp with F at position 6 or position 7, or both positions |
| | | 6 and 7 |
| | | F₁ = Phe with CF₃ at position 4 |
| | | L₁ = Leu with F, Cl, Br, I, CH₃, CF₃, CN, OH, or NO₂ at |
| | | positions γ, δ1 or δ2 |

*All amino acids are in the D-configuration.

TABLE 2
^{D}PMI-α based D-Peptide and Variant Antagonists of MDM2 and MDMX

| | SEQ | |
|---|---|---|
| Name | ID NO: | Amino Acid Sequence |
| ^{D}PMI-α | 35 | TNWYANLEKLLR |
| ^{D}PMI-α-del | 36 | WYANLEKLLR |
| ^{D}PMI-α-W₁ | 37 | TNW₁YANLEKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-α-W₁-del | 38 | W₁YANLEKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-α-L₁ | 39 | TNWYANL₁EKLLR |
| | | L₁ = Leu with F, Cl, Br, I, CH₃, CF₃, CN, OH, or NO₂ at |
| | | positions γ, δ1 or δ2 |
| ^{D}PMI-α-L₁-del | 40 | WYANL₁EKLLR |
| | | L₁ = Leu with F, Cl, Br, I, CH₃, CF₃, CN, OH, or NO₂ at |
| | | positions γ, δ1 or δ2 |
| ^{D}PMI-α-L₂ | 41 | TNWYANLEKLL₂R |
| | | L₂ = Leu with F, Cl, Br, I, CH₃, CF₃, CN, OH, or NO₂ at |
| | | positions γ, δ1 or δ2 |
| ^{D}PMI-α-L₂-del | 42 | WYANLEKLL₂R |
| | | L₂ = Leu with F, Cl, Br, I, CH₃, CF₃, CN, OH, or NO₂ at |
| | | positions γ, δ1 or δ2 |
| ^{D}PMI-α-WIL, | 43 | TNW₁YANL₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-α-W₁L₁-del | 44 | W₁YANL₁EKLLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-α-L₁L₂ | 45 | TNWYANL₁EKLL₂R |
| | | L₁ = Leu with F, Cl, Br, I, CH₃, CF₃, CN, OH, or NO₂ at |
| | | positions γ, δ1 or δ2 |
| | | L₂ = Leu with F, Cl, Br, I, CH₃, CF₃, CN, OH, or NO₂ at |
| | | positions γ, δ1 or δ2 |
| ^{D}PMI-α-L₁L₂-del | 46 | WYANL₁EKLL₂R |
| | | L₁ = Leu with F, Cl, Br, I, CH₃, CF₃, CN, OH, or NO₂ at |
| | | positions γ, δ1 or δ2 |
| | | L₂ = Leu with F, Cl, Br, I, CH₃, CF₃, CN, OH, or NO₂ at |
| | | positions γ, δ1 or δ2 |
| ^{D}PMI-α-W₁L₁L₂ | 47 | TNW₁YANL₁EKLL₂R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| | | L₂ = Leu with F, Cl, Br, I, CH₃, CF₃, CN, OH, or NO₂ at |
| | | positions γ, δ1 or δ2 |
| ^{D}PMI-α-W₁L₁L₂-del | 48 | W₁YANL₁EKLL₂R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| | | L₂ = Leu with F, Cl, Br, I, CH₃, CF₃, CN, OH, or NO₂ at |
| | | positions γ, δ1 or δ2 |

*All amino acids are in the D-configuration.

TABLE 3
^{D}MPI-γ based D-Peptide and Variant Antagonists of MDM2 and MDMX

| | SEQ | |
|---|---|---|
| Name | ID NO: | Amino Acid Sequence |
| ^{D}PMI-γ | 49 | DWWPLAFEALLR |
| ^{D}PMI-γ-del | 50 | WPLAFEALLR |
| ^{D}PM1-γ-3W₁ | 51 | DWW₁PLAFEALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-γ-3W₁-del | 52 | W₁PLAFEALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-γ-F₁ | 53 | DWWPLAF₁EALLR |
| | | F₁ =Phe with F, Cl, Br, I, CH₃, CF₃, CN, OH, or NO₂ at |
| | | position 4 |
| ^{D}PMI-γ-F₁-del | 54 | WPLAF₁EALLR |
| | | F₁ = Phe with F, Cl, Br, I, CH₃, CF₃, CN, OH, or NO₂ at |
| | | position 4 |
| ^{D}PMI-γ-L₁ | 55 | DWWPLAFEALL₁R |
| | | L₁ = Leu with F, Cl, Br, I, CH₃, CF₃, CN, OH, or NO₂ at |
| | | positions γ, δ1 or δ2 |
| ^{D}PMI-γ-L₁-del | 56 | WPLAFEALL₁R |
| | | L₁ = Leu with F, Cl, Br, I, CH₃, CF₃, CN, OH, or NO₂ at |
| | | positions γ, δ1 or δ2 |
| ^{D}PMI-γ-3W₁F₁ | 57 | DWW₁PLAF₁EALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-γ-3W₁F₁-del | 58 | W₁PLAF₁EALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-γ-F₁L₁ | 59 | DWWPLAF₁EALL₁R |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-γ-F₁L₁-del | 60 | WPLAF₁EALL₁R |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-γ-3W₁F¹L₁ | 61 | DWW₁PLAF₁EALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-γ-3W₁F₁L₁-del | 62 | W₁PLAF₁EALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |
| ^{D}PMI-γ-3W₁L₁ | 92 | DWW₁PLAFEALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-γ-3W₁L₁-del | 93 | W₁PLAFEALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-γ-2W₁ | 94 | DW₁WPLAFEALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-γ-2W₁F₁ | 95 | DW₁WPLAF₁EALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-γ-2W₁L₁ | 96 | DW₁WPLAFEALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-γ-2W₁F₁L₁ | 97 | DW₁WPLAF₁EALL₁R |
| | | W1 = Trp with F, Cl, Br, I, CH₃, CF₃, CN, OH, or NO₂ at |
| | | position 6 or position 7, or both positions 6 and 7 where the |
| | | side group is the same or different |
| | | F1 = Phe with F, Cl, Br, I, CH₃, CF₃, CN, OH, or NO₂ at |
| | | position 4 |
| | | L1 = Leu with F, Cl, Br, I, CH₃, CF₃, CN, OH, or NO₂ at |
| | | positions γ, δ1 or δ2 |
| ^{D}PMI-γ-2W₁3W₁ | 98 | DW₁W₂PLAFEALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | W₂ = Trp with F, Cl, Br, I, CH₃, CF₃, CN, OH, or NO₂ at |
| | | position 6 or position 7, or both positions 6 and 7 where the |
| | | side group is the same or different |
| ^{D}PMI-γ-2W₁3W₁F₁ | 99 | DW₁W₂PLAF₁EALLR |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | W₂ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| ^{D}PMI-γ-2W₁3W₁L₁ | 100 | DW₁W₂PLAFEALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | W₂ = Trp with F, Cl, Br, I, CH₃, CF₃, CN, OH, or NO₂ at |
| | | position 6 or position 7, or both positions 6 and 7 where the |
| | | side group is the same or different |
| | | L₁ = Leu with F, Cl, Br, I, CH₃, CF₃, CN, OH, or NO₂ at |
| | | positions γ, δ1 or δ2 |
| ^{D}PMI-γ-2W₁3W₁F₁L₁ | 101 | DW₁WPLAF₁EALL₁R |
| | | W₁ = Trp with F, Cl, Br, I, CH₃, CF₃, CN, OH, or NO₂ at |
| | | position 6 or position 7, or both positions 6 and 7 where the |
| | | side group is the same or different |
| | | W₂ = Trp with F, Cl, Br, I, CH₃, CF₃, CN, OH, or NO₂ at |
| | | position 6 or position 7, or both positions 6 and 7 where the |
| | | side group is the same or different |
| | | F₁ = Phe with F, Cl, Br, I, CH₃, CF₃, CN, OH, or NO₂ at |
| | | position 4 |
| | | L₁ = Leu with F, Cl, Br, I, CH₃, CF₃, CN, OH, or NO₂ at |
| | | positions γ, δ1 or δ2 |

*All amino acids are in the D-configuration.

TABLE 4
Stapled D-Peptide and Variant Antagonists of MDM2 and MDMX
A.

| | |
|---|---|
| 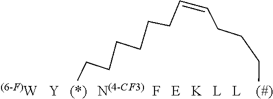 | 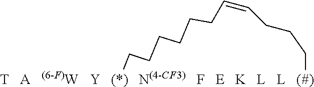 |
| 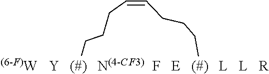 | 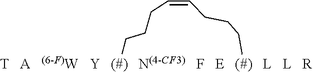 |
| 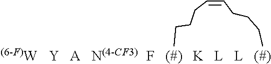 | 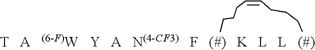 |
| 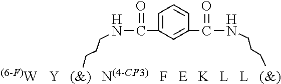 | 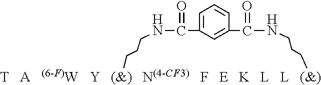 |
| 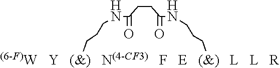 | 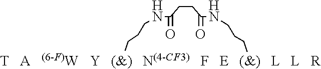 |

The symbols *, # and & are defined below in Table 4B. The structures
above correspond to the following names and SEQ ID NOs.

| | |
|---|---|
| ^{D}PMI-δ-A; SEQ ID NO: 64 | ^{D}PMI-δ-B; SEQ ID NO: 65 |
| ^{D}PMI-δ-C; SEQ ID NO: 66 | ^{D}PMI-δ-D; SEQ ID NO: 67 |
| ^{D}PMI-δ-E; SEQ ID NO: 68 | ^{D}PMI-δ-F; SEQ ID NO: 69 |
| ^{D}PMI-δ-G; SEQ ID NO: 70 | ^{D}PMI-δ-H; SEQ ID NO: 71 |
| ^{D}PMI-δ-I; SEQ ID NO: 72 | ^{D}PMI-δ-J; SEQ ID NO: 73 |

B.

| | |
|---|---|
| 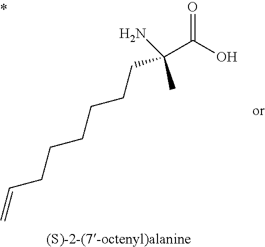 | 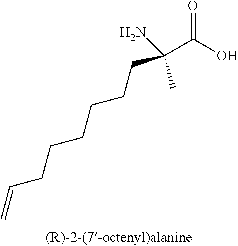 |
| 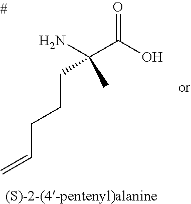 | 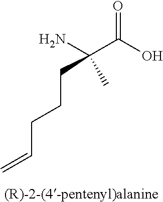 |
| 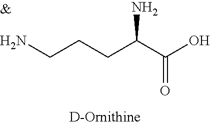 | |

TABLE 5
Stapled D-Peptide and Variant Antagonists of MDM2 and MDMX
A. ^{D}PMI-γ stapled peptides, where O is an amino acid with an olefinic side
chain, and m and n are each independently an integer between 1 and 6.

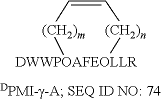

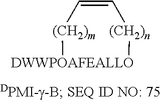

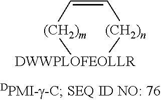

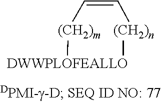

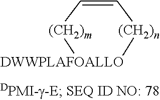

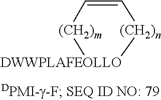

B. ^{D}PMI-δ stapled peptides, where O is an amino acid with an olefinic side
chain, and m and n are each independently an integer between 1 and 6.

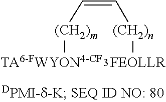

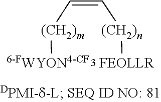

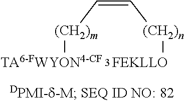

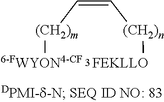

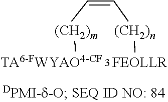

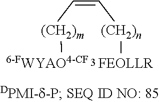

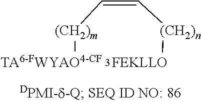

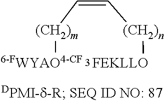

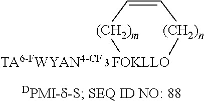

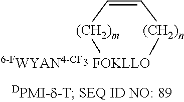

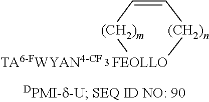

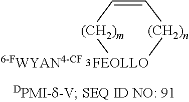

### High Affinity

The MDM2/MDMX antagonists of the present invention bind MDM2 or MDMX, or both MDM2 and MDMX, with high affinity. If antagonist does not bind both MDM2 and MDMX with high affinity, it may bind one of the proteins with low affinity, or be found to have no measurably affinity for one of the proteins. As used herein, “high affinity” means a K_{D} value in the nanomolar range or lower, such as less than about 1 nM, 5 nM, 10 nM, 50 nM, 100 nM, 60 nM, 80 nM, 100 nM, 120 nM, 140 nM, 160 nM, 180 nM, 200 nM, 220 nM, 240 nM, 260 nM, 280 nM, 300 nM, 320 nM, 340 nM, 360 nM, 380 nM, 400 nM, 420 nM, 440 nM, 460 nM, 480 nM, or 500 nM, or less. As described herein, some of the antagonists have very high affinity for one or both of MDM2 and MDMX, with a K_{D} value in the picomolar range, such as less than about 1 pM, 5 pM, 10 pM, 50 pM, 100 pM, 60 pM, 80 pM, 100 pM, 120 pM, 140 pM, 160 pM, 180 pM, 200 pM, 220 pM, 240 pM, 260 pM, 280 pM, 300 pM, 320 pM, 340 pM, 360 pM, 380 pM, 400 pM, 420 pM, 440 pM, 460 pM, 480 pM, or 500 pM, or less. The high affinity of the MDM2/MDMX antagonists may also be understood in terms of a range, and includes a K_{D} value from about 10 to 500 nM, from about 20 to 400 nM, from about 25 to 300 nM, from about 30 to 220 nM, from about 30 to 220 nM, and from about 10 to 500 pM, from about 20 to 400 pM, from about 25 to 300 pM, from about 30 to 220 pM, from about 30 to 220 pM.

The MDM2/MDMX antagonists of the present invention preferably bind to MDM2 in the p53 binding pocket, which is located at the N-terminus of MDM2 and encompasses, approximately, amino acids 17-124 of the MDM2 amino acid sequence. See Kussie, P. H., et al. Science, 274: 948-953 (1996). More specifically, the p53 binding pocket is encompassed by amino acids 25-109 of the full length sequence.

The MDM2/MDMX antagonists of the present invention also preferably bind to MDMX in the p53 binding pocket, which is located at the N-terminus of MDMX and encompasses, approximately, amino acids 1-185 of the MDMX amino acid sequence. See Böttger, V. A., et al., Oncogene 18(1):189-199 (1999). More specifically, the p53 binding pocket is encompassed by amino acids 24-108 of the full length sequence.

The present invention also encompasses: (i) polynucleotide sequences encoding MDM2/MDMX antagonists of the present invention, (ii) vectors into which the polynucleotide sequences are inserted, (iii) host cells genetically engineered (transduced, transformed, or transfected) with the vectors, (iv) methods of culturing the host cells under conditions promoting production of the MDM2/MDMX antagonists encoded by the polynucleotide sequences, and (v) methods of isolating the expressed MDM2/MDMX antagonists from the culture media and host cells.

The polynucleotide sequences of the present invention may be in the form of RNA or DNA, which DNA includes cDNA, genomic DNA, and synthetic DNA. The DNA may be double-stranded or single-stranded, and if single stranded may be the coding strand or non-coding (anti-sense) strand. The coding sequence which encodes the peptides, derivatives and variants may vary due to the redundancy or degeneracy of the genetic code, yet encode the same peptide.

The present invention also includes polynucleotide sequences wherein the coding sequence for the peptide may be fused in the same reading frame to a polynucleotide which encodes a peptide or protein that aids in expression and secretion of a polypeptide from a host cell, for example, a leader sequence which functions as a secretory sequence for controlling transport of a peptide from the cell. The leader sequence is cleaved by the host cell to form the mature form of the MDM2/MDMX antagonist.

The polynucleotide sequences of the present invention may also have the coding sequence fused in frame to a marker sequence which allows for purification of an antagonist of the present invention. The marker sequence may be, but is not limited to, a hexa-histidine tag supplied by a pQE-9 vector to provide for purification of the mature peptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell 37:767 (1984)).

The vector into which the polynucleotide sequence is inserted may any one of a variety of expression vectors for expressing a polypeptide. Such vectors include chromosomal, non-chromosomal and synthetic DNA sequences, e.g., derivatives of SV40, bacterial plasmids, phage DNA, yeast plasmids, vectors derived from combinations of plasmids and phage DNA, viral DNA such as vaccinia, adenovirus, fowl pox virus, and pseudorabies. Any other plasmid or vector may be used so long as it is replicable and viable in the host cell. The vector containing the polynucleotide sequence may contain an appropriate promoter or control sequence. The vector may contain at least one selectable marker gene to provide a phenotypic trait for selection of transformed host cells. Such markers include dihydrofolate reductase (DHFR) or neomycin resistance for eukaryotic cell culture, and tetracycline or ampicillin resistance for culturing in E. coli and other bacteria. Representative examples of appropriate host cells, include but are not limited to: bacterial cells, such as E. coli, Salmonella typhimurium, fungal cells, such as yeast, insect cells, such as Drosophila S2 and Spodoptera Sf19, animal cells such as CHO, COS, and Bowes melanoma; and plant cells.

The antagonists can be recovered and purified from host cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. It is preferred to have low concentrations (approximately 0.1 15 mM) of calcium ion present during purification (Price et al., J. Biol. Chem. 244:917 (1969)). Protein refolding steps can be used, as necessary, in completing configuration of the mature protein. High performance liquid chromatography (HPLC) can be employed for final purification steps.

The MDM2/MDMX antagonists of the invention may also be synthetically produced by conventional peptide synthesizers. See, Creighton, 1983, Proteins: Structures and Molecular Principles, W. H. Freeman & Co., N.Y., and Hunkapiller, M., et al., 1984, Nature 310:105-111. Furthermore, such techniques allow the introduction of non-classical amino acids or chemical amino acid analogs into the peptides, thus producing the variants of the present invention. Non-classical amino acids include, but are not limited to, the D-amino acids, 2,4-diaminobutyric acid, a-amino isobutyric acid, 4-aminobutyric acid, Abu, 2-amino butyric acid, g-Abu, e-Ahx, 6-amino hexanoic acid, Aib, 2-amino isobutyric acid, 3-amino propionic acid, ornithine, norleucine, norvaline, hydroxyproline, sarcosine, citrulline, homocitrulline, cysteic acid, t-butylglycine, t-butylalanine, phenylglycine, cyclohexylalanine, b-alanine, fluoro-amino acids, designer amino acids such as b-methyl amino acids, Ca-methyl amino acids, Na-methyl amino acids, and amino acid analogs in general, and unnatural amino acids with olefinic side chains. Specific examples include (S)-2-(7′-octenyl)alanine, (R)-2-(7′-octenyl)alanine, (S)-2-(4′-pentenyl)alanine, (R)-2-(4′-pentenyl)alanine, and D-ornithine, each of which is used in the production of the stapled D-peptide shown in Table 4A.

The invention encompasses MDM2/MDMX antagonists that are modified during or after translation, e.g., by glycosylation, acetylation, phosphorylation, amidation, derivatization by known protecting/blocking groups, proteolytic cleavage, linkage to an antibody molecule or other cellular ligand, etc. Any of numerous chemical modifications may be carried out by known techniques, including but not limited, to specific chemical cleavage by cyanogen bromide, trypsin, chymotrypsin, papain, V8 protease, NaBH₄; acetylation, formylation, oxidation, reduction; metabolic synthesis in the presence of tunicamycin, etc.

Additional post-translational modifications encompassed by the invention include, e.g., N-linked or O-linked carbohydrate chains, processing of N-terminal or C-terminal ends, attachment of chemical moieties to the amino acid backbone, chemical modifications of N-linked or O-linked carbohydrate chains, and addition or deletion of an N-terminal methionine residue as a result of prokaryotic host cell expression. The MDM2/MDMX antagonists may also be modified with a detectable label, such as an enzymatic, fluorescent, isotopic or affinity label to allow for detection and isolation of the peptide.

### Pharmaceutical Compositions

The present invention also relates to pharmaceutical compositions comprising the MDM2/MDMX antagonists of the present invention. The MDM2/MDMX antagonists may be used in combination with any suitable pharmaceutical excipient or carrier. Such pharmaceutical compositions comprise a therapeutically effective amount of one or more MDM2/MDMX antagonist, and pharmaceutically acceptable excipient(s) and/or carrier(s). The specific formulation will suit the mode of administration.

Excipients included in the pharmaceutical compositions have different purposes depending, for example on the nature of the drug, and the mode of administration. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, lubricating agents (such as talc or silica, and fats, such as vegetable stearin, magnesium stearate or stearic acid), emulsifiers, suspending or viscosity agents, inert diluents, fillers (such as cellulose, dibasic calcium phosphate, vegetable fats and oils, lactose, sucrose, glucose, mannitol, sorbitol, calcium carbonate, and magnesium stearate), disintegrating agents (such as crosslinked polyvinyl pyrrolidone, sodium starch glycolate, cross-linked sodium carboxymethyl cellulose), binding agents (such as starches, gelatin, cellulose, methyl cellulose or modified cellulose such as microcrystalline cellulose, hydroxypropyl cellulose, sugars such as sucrose and lactose, or sugar alcohols such as xylitol, sorbitol or maltitol, polyvinylpyrrolidone and polyethylene glycol), wetting agents, antibacterials, chelating agents, coatings (such as a cellulose film coating, synthetic polymers, shellac, corn protein zein or other polysaccharides, and gelatin), preservatives (including vitamin A, vitamin E, vitamin C, retinyl palmitate, and selenium, cysteine, methionine, citric acid and sodium citrate, and synthetic preservatives, including methyl paraben and propyl paraben), sweeteners, perfuming agents, flavoring agents, coloring agents, administration aids, and combinations thereof.

Carriers are compounds and substances that improve and/or prolong the delivery of an active ingredient to a subject in the context of a pharmaceutical composition. Carrier may serve to prolong the in vivo activity of a drug or slow the release of the drug in a subject, using controlled-release technologies. Carriers may also decrease drug metabolism in a subject and/or reduce the toxicity of the drug. Carrier can also be used to target the delivery of the drug to particular cells or tissues in a subject. Common carriers (both hydrophilic and hydrophobic carriers) include fat emulsions, lipids, PEGylated phospholids, PEGylated liposomes, PEGylated liposomes coated via a PEG spacer with a cyclic RGD peptide c(RGD^{D}YK), liposomes and lipospheres, microspheres (including those made of biodegradable polymers or albumin), polymer matrices, biocompatible polymers, protein-DNA complexes, protein conjugates, erythrocytes, vesicles, nanoparticles, and side-chains for hydro-carbon stapling. The aforementioned carriers can also be used to increase cell membrane permeability of the MDM2/MDMX antagonists of the invention. In addition to their use in the pharmaceutical compositions of the present invention, carriers may also be used in compositions for other uses, such as research uses in vitro (e.g., for delivery to cultured cells) and/or in vivo.

Pharmaceutical compositions adapted for oral administration may be presented as discrete units such as capsules or tablets; as powders or granules; as solutions, syrups or suspensions (in aqueous or non-aqueous liquids; or as edible foams or whips; or as emulsions). Suitable excipients for tablets or hard gelatine capsules include lactose, maize starch or derivatives thereof, stearic acid or salts thereof. Suitable excipients for use with soft gelatine capsules include for example vegetable oils, waxes, fats, semi-solid, or liquid polyols etc. For the preparation of solutions and syrups, excipients which may be used include for example water, polyols and sugars. For the preparation of suspensions oils, e.g. vegetable oils, may be used to provide oil-in-water or water in oil suspensions. In certain situations, delayed release preparations may be advantageous and compositions which can deliver the MDM2/MDMX antagonists in a delayed or controlled release manner may also be prepared. Prolonged gastric residence brings with it the problem of degradation by the enzymes present in the stomach and so enteric-coated capsules may also be prepared by standard techniques in the art where the active substance for release lower down in the gastro-intestinal tract.

Pharmaceutical compositions adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6):318 (1986).

Pharmaceutical compositions adapted for topical administration may be formulated as ointments, creams, suspensions, lotions, powders, solutions, pastes, gels, sprays, aerosols or oils. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes.

Pharmaceutical compositions adapted for rectal administration may be presented as suppositories or enemas.

Pharmaceutical compositions adapted for nasal administration wherein the carrier is a solid include a coarse powder having a particle size for example in the range 20 to 500 microns which is administered in the manner in which snuff is taken, i.e., by rapid inhalation through the nasal passage from a container of the powder held close up to the nose. Suitable compositions wherein the carrier is a liquid, for administration as a nasal spray or as nasal drops, include aqueous or oil solutions of the active ingredient.

Pharmaceutical compositions adapted for administration by inhalation include fine particle dusts or mists which may be generated by means of various types of metered dose pressurised aerosols, nebulizers or insufflators.

Pharmaceutical compositions adapted for vaginal administration may be presented as pessaries, tampons, creams, gels, pastes, foams or spray formulations.

Pharmaceutical compositions adapted for parenteral administration include aqueous and non-aqueous sterile injection solution which may contain anti-oxidants, buffers, bacteriostats and solutes which render the formulation substantially isotonic with the blood of the intended recipient; and aqueous and non-aqueous sterile suspensions which may include suspending agents and thickening agents. Excipients which may be used for injectable solutions include water-for-injection, alcohols, polyols, glycerine and vegetable oils, for example. The compositions may be presented in unit-dose or multi-dose containers, for example sealed ampoules and vials, and may be stored in a freeze-dried (lyophilized) condition requiring only the addition of the sterile liquid carrier, for example water or saline for injections, immediately prior to use. Extemporaneous injection solutions and suspensions may be prepared from sterile powders, granules and tablets. The pharmaceutical compositions may contain preserving agents, solubilising agents, stabilising agents, wetting agents, emulsifiers, sweeteners, colourants, odorants, salts (substances of the present invention may themselves be provided in the form of a pharmaceutically acceptable salt), buffers, coating agents or antioxidants. They may also contain therapeutically-active agents in addition to the substance of the present invention.

The pharmaceutical compositions may be administered in a convenient manner such as by the topical, intravenous, intraperitoneal, intramuscular, intratumor, subcutaneous, intranasal or intradermal routes. The pharmaceutical compositions are administered in an amount which is effective for treating and/or prophylaxis of the specific indication. In general, the pharmaceutical compositions are administered in an amount of at least about 0.1 mg/kg to about 100 mg/kg body weight. In most cases, the dosage is from about 10 mg/kg to about 1 mg/kg body weight daily, taking into account the routes of administration, symptoms, etc.

Dosages of the MDM2/MDMX antagonists of the present invention can vary between wide limits, depending upon the location, source, identity, extent and severity of the cancer, the age and condition of the individual to be treated, etc. A physician will ultimately determine appropriate dosages to be used.

As used herein, the term “administer” and “administering” are used to mean introducing at least one MDM2/MDMX antagonist, or a pharmaceutical composition comprising at least one MDM2/MDMX antagonist, into a subject. When administration is for the purpose of treatment, the substance is provided at, or after the diagnosis of an abnormal cell growth, such as a tumor. The therapeutic administration of this substance serves to inhibit cell growth of the tumor or abnormal cell growth.

As used herein, the term “co-administer” means that each of at least two different biological active compounds are administered to a subject during a time frame wherein the respective periods of biological activity overlap. Thus, the term includes sequential as well as co-extensive administration. When co-administration is used, the routes of administration need not be the same. The biological active compounds include MDM2/MDMX antagonists, as well as other compounds useful in treating cancer, including but not limited to agents such as vinca alkaloids, nucleic acid inhibitors, platinum agents, interleukin-2, interferons, alkylating agents, antimetabolites, corticosteroids, DNA intercalating agents, anthracyclines, and ureas. Examples of specific agents in addition to those exemplified herein, include hydroxyurea, 5-fluorouracil, anthramycin, asparaginase, bleomycin, dactinomycin, dacabazine, cytarabine, busulfan, thiotepa, lomustine, mechlorehamine, cyclophosphamide, melphalan, mechlorethamine, chlorambucil, carmustine, 6-thioguanine, methotrexate, etc. The skilled artisan will understand that two different MDM2/MDMX antagonists may be co-administered to a subject, or that a MDM2/MDMX antagonist and an agent, such as one of the agents provided above, may be co-administered to a subject.

As used herein, the terms “dose”, “dosage”, “unit dose”, “unit dosage”, “effective dose” and related terms refer to physically discrete units that contain a predetermined quantity of active ingredient (e.g., MDM2/MDMX antagonist) calculated to produce a desired therapeutic effect (e.g., death of cancer cells). These terms are synonymous with the therapeutically-effective amounts and amounts sufficient to achieve the stated goals of the methods disclosed herein.

As used herein, the terms “treat”, “treating”, and “treatment” have their ordinary and customary meanings, and include one or more of: blocking, ameliorating, or decreasing in severity and/or frequency a symptom of cancer in a subject, and/or inhibiting the growth, division, spread, or proliferation of cancer cells, or progression of cancer (e.g., emergence of new tumors) in a subject. Treatment means blocking, ameliorating, decreasing, or inhibiting by about 1% to about 100% versus a subject to which a MDM2/MDMX antagonist has not been administered. Preferably, the blocking, ameliorating, decreasing, or inhibiting is about 100%, 99%, 98%, 97%, 96%, 95%, 90%, 80%, 70%, 60%, 50%, 40%, 30%, 20%, 10%, 5% or 1% versus a subject to which a MDM2/MDMX antagonist has not been administered.

The MDM2/MDMX antagonists may also be employed in accordance with the present invention by expression of the antagonists in vivo, i.e., via gene therapy. The use of the peptides or compositions in a gene therapy setting is also considered to be a type of “administration” of the peptides for the purposes of the present invention.

Accordingly, the present invention also relates to methods of treating a subject having cancer, comprising administering to the subject a pharmaceutically-effective amount of one or more MDM2/MDMX antagonist of the present invention, or a pharmaceutical composition comprising one or more of the antagonists to a subject needing treatment. The term “cancer” is intended to be broadly interpreted and it encompasses all aspects of abnormal cell growth and/or cell division. Examples include: carcinoma, including but not limited to adenocarcinoma, squamous cell carcinoma, adenosquamous carcinoma, anaplastic carcinoma, large cell carcinoma, small cell carcinoma, and cancer of the skin, breast, prostate, bladder, vagina, cervix, uterus, liver, kidney, pancreas, spleen, lung, trachea, bronchi, colon, small intestine, stomach, esophagus, gall bladder; sarcoma, including but not limited to chondrosarcoma, Ewing's sarcoma, malignant hemangioendothelioma, malignant schwannoma, osteosarcoma, soft tissue sarcoma, and cancers of bone, cartilage, fat, muscle, vascular, and hematopoietic tissues; lymphoma and leukemia, including but not limited to mature B cell neoplasms, such as chronic lymphocytic leukemia/small lymphocytic lymphoma, B-cell prolymphocytic leukemia, lymphomas, and plasma cell neoplasms, mature T cell and natural killer (NK) cell neoplasms, such as T cell prolymphocytic leukemia, T cell large granular lymphocytic leukemia, aggressive NK cell leukemia, and adult T cell leukemia/lymphoma, Hodgkin lymphomas, and immunodeficiency-associated lymphoproliferative disorders; germ cell tumors, including but not limited to testicular and ovarian cancer; blastoma, including but not limited to hepatoblastoma, medulloblastoma, nephroblastoma, neuroblastoma, pancreatoblastoma, leuropulmonary blastoma and retinoblastoma. The term also encompasses benign tumors.

In each of the embodiments of the present invention, the subject receiving treatment is a human or non-human animal, e.g., a non-human primate, bird, horse, cow, goat, sheep, a companion animal, such as a dog, cat or rodent, or other mammal. In some embodiments, the subject is a human.

The invention also provides a kit comprising one or more containers filled with one or more of the ingredients of the pharmaceutical compositions of the invention, such as a container filled with a pharmaceutical composition comprising a MDM2/MDMX antagonist and a carrier or diluent. Associated with such container(s) can be a notice in the form prescribed by a governmental agency regulating the manufacture, use or sale of pharmaceuticals or biological products, which notice reflects approval by the agency of manufacture, use or sale for human administration. In addition, the pharmaceutical compositions may be employed in conjunction with other therapeutic compounds.

The examples disclosed herein are intended to be illustrative of select embodiments of the present invention and not meant to limit the scope of the invention in any manner.

### EXAMPLES

### Example 1

### Identification of Potent D-Peptide Inhibitors of the p53-MDM2 Interaction by Mirror Image Phage Display

Previously, a potent dual-specificity L-peptide inhibitor of the p53-MDM2/MDMX interactions was identified via phage display and structurally characterized (Pazgier M, et al. Proc Natl Acad Sci USA 106(12):4665-70 (2009)). The duodecimal peptide, termed PMI (TSFAEYWNLLSP; SEQ ID NO:63), bound the p53-binding domains of MDM2 (²⁵⁻¹⁰⁹MDM2 or ^{syn}MDM2) and MDMX (²⁴⁻¹⁰⁸MDMX or syn MDMX) at affinities of 3.2 and 8.5 nM, respectively, as determined by surface plasmon resonance (SPR) and isothermal titration calorimetry (ITC) techniques (Pazgier M, et al. Proc Natl Acad Sci USA 106(12):4665-70 (2009); Li C, et al. Angew Chem Int Ed Engl 48(46):8712-5 (2009); Li C, et al. J Mol Biol 398(2):200-13 (2010)). A single mutation, N8A, turned PMI into one of the most potent dual-specificity inhibitors of the p53-MDM2/MDMX interactions reported to date, registering respective K_{D} values of 490 pM and 2.4 nM for MDM2 and MDMX (Li C, et al. J Mol Biol 398(2):200-13 (2010)). Structural analysis indicated that high-affinity peptide binding to MDM2 and MDMX necessitated, in addition to optimized inter-molecular interactions, enhanced helix stability or propensity contributed by non-contact residues (Li C, et al. J Mol Biol 398(2):200-13 (2010)).

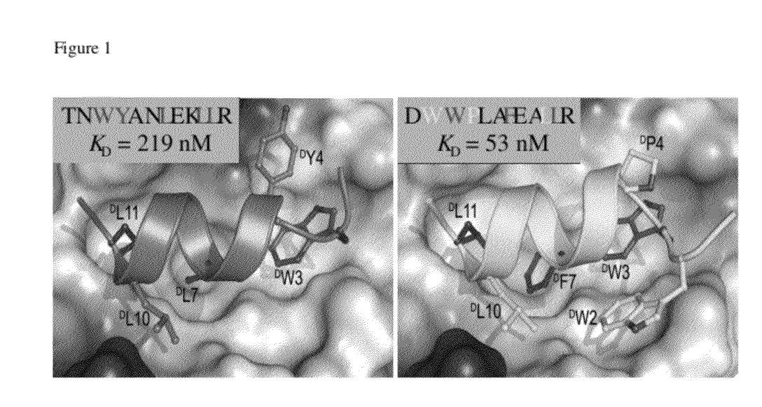

For mirror image phage display (Eckert D M, et al. Cell 99(1):103-15 (1999); Schumacher T N, et al. Science 271(5257):1854-7 (1996)), the D-enantiomeric form of ^{syn}MDM2 composed entirely of D-amino acids was chemically synthesized via native chemical ligation (Dawson P E and Kent S B. Annu Rev Biochem 69:923-60 (2000); Dawson P E, et al. Science 266(5186):776-9 (1994)), and site-specifically biotinylated for streptavidin capturing. A strong consensus binding sequence TNWYANLEKLLR (SEQ ID NO:35) was obtained (Liu M, et al. Angew Chem Int Ed Engl 49(21):3649-3652 (2010)). The binding affinity of its D-enantiomer, termed ^{D}PMI-α, was quantified for ^{syn}MDM2 and ^{syn}MDMX by competition SPR, yielding K_{D} values of 219 nM and 18 μM, respectively. A double mutation N2A/L7F resulted in an inhibitor termed ^{D}PMI-β (TAWYANFEKLLR; SEQ ID NO:1), which bound ^{syn}MDM2 and ^{syn}MDMX at affinities of 34.5 nM and 2.4 μM, respectively (Liu M, et al. Angew Chem Int Ed Engl 49(21):3649-3652 (2010)). When phage display was performed under a more stringent selection condition, a distinct consensus sequence ensued (DWWPLAFEALLR; SEQ ID NO:49) (Liu M, et al. Proc Natl Acad Sci USA 107(32):14321-6 (2010)). Its D-enantiomer, termed ^{D}PMI-γ, bound ^{syn}MDM2 and ^{syn}MDMX with respective K_{D} values of 52.8 nM and 4.9 μM. Unlike L-peptides that generally do not display strong selectivity toward either MDM2 or MDMX binding (Li C, et al. J Mol Biol 398(2):200-13 (2010)), all the D-peptide ligands bound MDM2 approximately two orders of magnitude stronger than they did MDMX. As expected, both ^{D}PMI-α and ^{D}PMI-γ adopt a distinct left-handed helical conformation in the complex with ^{syn}MDM2, docking critical hydrophobic side chains inside the p53-binding pocket (FIG. 1).

### Example 2

### In Vitro and In Vivo Studies of D-Peptide Inhibitors

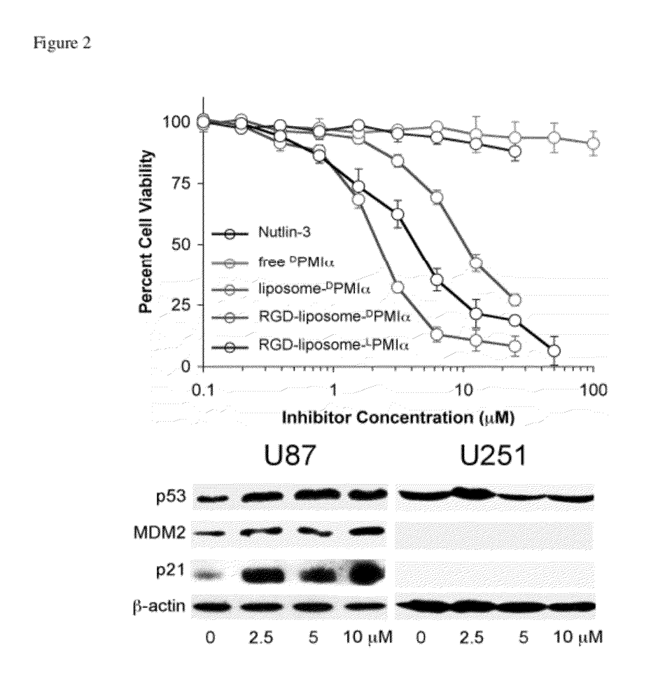

Each of the D-peptides is fully resistant to proteolysis (Liu M, et al. Proc Natl Acad Sci USA 107(32):14321-6 (2010); Liu M, et al. Angew Chem Int Ed Engl 49(21):3649-3652 (2010)). To increase cell-membrane permeability of the D-peptides, PEGylated liposomes were designed as a carrier vehicles to encapsulate the D-peptides. The encapsulated D-peptides were evaluated for their anti-tumor activity in vitro and therapeutic efficacy in vivo. As integrin avβ3 is highly expressed on the surface of glioma tumor cells (Desgrosellier J S and Cheresh D A. Nat Rev Cancer 10(1):9-22 (2010); Gladson C L and Cheresh D A. J Clin Invest 88(6):1924-32 (1991)), the tumor model for this study, therefore liposomes were also coated via a PEG spacer with a cyclic RGD peptide c(RGD^{D}YK) to facilitate tumor-specific targeting. As shown in FIG. 2, ^{D}PMI-α induced a dose-dependent growth inhibition of human glioma cell line U87 (wild type p53) with an IC₅₀ value of 1.9 μM as determined by the MTS cell viability assay, more effective than Nutlin-3 (IC₅₀=3.8 μM). In sharp contrast, human glioma U251 cells (mutant p53) were largely resistant to the treatment of RGD-liposome-^{D}PMI-α (Liu M, et al. Proc Natl Acad Sci USA 107(32):14321-6 (2010)), suggesting that ^{D}PMI-α functioned in a p53-dependent manner. Consistent with this finding, Western blotting analysis showed dose-dependent expression of the p53 responsive genes MDM2 and p2.1 in U87 cells but not in U251 cells following ^{D}PMI-α treatment (FIG. 2), supporting that intracellular ^{D}PMI-α inhibited U87 cell growth in vitro by reactivating the p53 pathway.

Importantly, in vivo therapeutic efficacy of RGD-liposome-^{D}PMI-α on U87 xenografts was clearly established in two different brain tumor models based on sites of inoculation—subcutaneous and intracranial. In the first animal model, treatment by RGD-liposome-^{D}PMI-α at a high dose completely inhibited tumor growth with no sighs of weight loss observed (Liu M, et al. Proc Natl Acad Sci USA 107(32):14321-6 (2010)). In the second animal model, RGD-liposome-^{D}PMI-α treatment significantly prolonged the survival of intracranial glioblastoma-bearing mice from an average survival time of 22.5 days (untreated) to 28 days at high doses (Liu M, et al. Proc Natl Acad Sci USA 107(32):14321-6 (2010). ^{D}PMI-β was found more effective than ^{D}PMI-α in similar in vitro and in vivo experiments (unpublished results). Taken together, these studies demonstrated that D-peptide antagonists of MDM2 efficiently kill tumor cells harboring wild type TP53 in cell cultures and experimental animals by re-activating the p53 pathway (Liu M, et al. Proc Natl Acad Sci USA 107(32):14321-6 (2010)), thus validating D-peptide antagonism of MDM2 as an attractive therapeutic paradigm for cancer treatment.

### Example 3

### Functional Optimization of ^{D}PMI-β

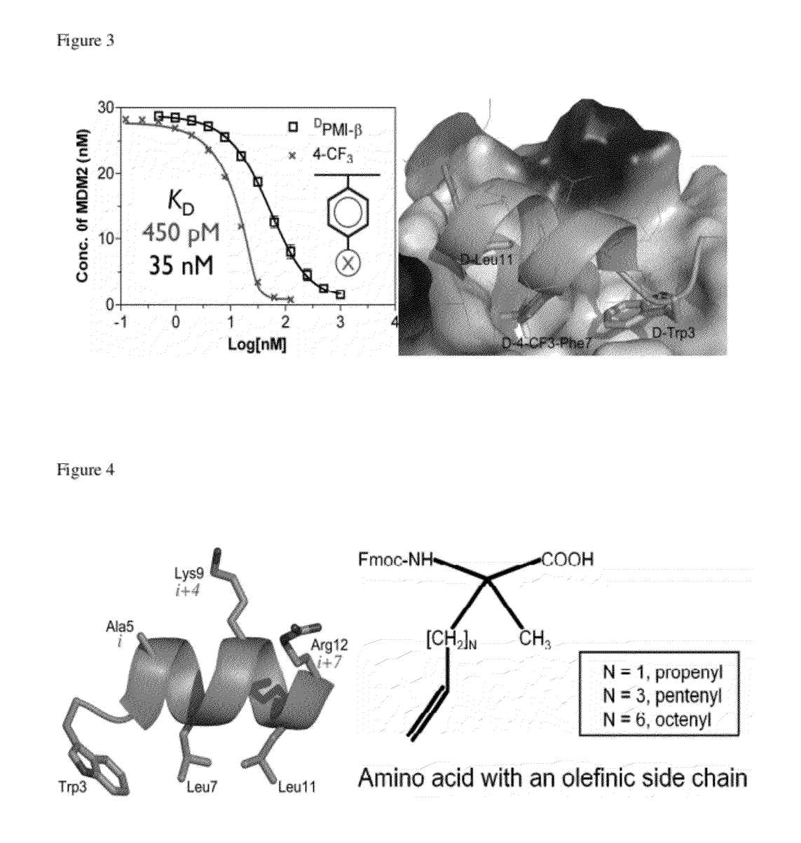

Structural analysis of MDM2 complexed with ^{D}PMI-β and ^{D}PMI-γ indicates that an unfilled space would exist in the binding pocket of Phe7 of ^{D}PMI-β despite the fact ^{D}PMI-β is the strongest ligand of MDM2 among the three. In the absence of a crystal structure of ^{D}PMI-β-MDM2, Phe7 was modified with 4-X-Phe and it was found that 4-X-Phe7-^{D}PMI-β binding to MDM2 improved when X=F, Cl, Br, I, CH₃, CF₃ or CN and weakened when X=OH or NO₂. Remarkably, substitution of 4-CF₃-Phe for Phe7 dramatically enhanced the binding affinity of _{D}PMI-β for MDM2 by ˜80-fold as measured by competition SPR (FIG. 3), consistent with the change of IC₅₀ values determined in a fluorescence polarization (FP)-based competition assay. The crystal structure of 4-CF₃-Phe7-^{D}PMI-β in complex with MDM2 was determined at 1.75 Å resolution (FIG. 3). MDM2-bound 4-CF₃-Phe7-^{D}PMI-β and ^{D}PMI-α are nearly identical, structurally validating the experimental design. Notably, a modest 2-fold improvement in binding affinity for MDM2 was observed upon substitution of 6-F-Trp for Trp3. In combination, both changes additively enhanced ^{D}PMI-β binding to MDM2, resulting in an overall decrease in K_{D} by ˜160-fold (K_{D}=220 pM). The resultant D-peptide, TA(6-F)WYAN(4-CF3)FEKLLR termed ^{D}PMI-δ, bound MDMX with a K_{D} value of 200 nM—substantially less potent than MDM2 binding. Nevertheless, ^{D}PMI-δ is still a moderately strong MDMX antagonist in its own right. It should be noted that chlorination of a critical Trp residue has been reported to dramatically increase the binding affinity of several peptidomimetic antagonists of MDM2 due to improved van der Waals interactions (Garcia-Echeverria C, et al. J Med Chem 43(17):3205-8 (2000); Grasslin A, et al. Chembiochem 10(8):1360-8 (2009); Kallen J, et al. J Biol Chem 284(13):8812-21 (2009); Sakurai K, et al. J Am Chem Soc 128(34):11000-1 (2006)).

### Example 4

### Design of Side-Chain Cross-Linked Forms of ^{D}PMI-δ Capable of Traversing the Cell Membrane to Stabilize Intracellular p53

Rationale.

Peptide antagonists of MDM2/MDMX must traverse the cell membrane to activate p53. In the absence of a suitable delivery vehicle, however, cellular uptake of peptides is generally inefficient—constituting a major functional obstacle that limits their therapeutic value.

An exceedingly potent 2^{nd} generation D-peptide inhibitor (^{D}PMI-δ) of the p53-MDM2 interaction has been successfully designed that is fully resistant to proteolysis. To fulfill its therapeutic potential, ^{D}PMI-δ must been endowed with the ability to traverse the cell membrane. Arg-rich cell-penetrating peptides (CPPs) are commonly used to promote efficient cellular uptake of covalently attached proteins and peptides to the cytoplasm and nucleus of many cell types (Brooks H, et al. Adv Drug Deliv Rev 57(4):559-77 (2005); Jones S W, et al. Br J Pharmacol 145(8):1093-102 (2005); Torchilin V P, et al. Proc Natl Acad Sci USA 98(15):8786-91 (2001); Vives E, et al. Biochim Biophys Acta. 1786(2):126-38 (2008); Wadia J S and Dowdy S F. Adv Drug Deliv Rev 57(4):579-96 (2005); Fittipaldi A and Giacca M. Adv Drug Deliv Rev 57(4):597-608 (2005)). However, it has been found that cationic CPP-conjugated hydrophobic peptides such as PMI act like detergents, cause membrane leakage and necrotic cell death, and are indiscriminately cytotoxic independently of p53 status (Liu M, et al. Angew Chem Int Ed Engl 49(21):3649-3652 (2010); Li C, et al. Angew Chem Mt Ed Engl 48(46):8712-5 (2009); Bowne W B, et al. Ann Surg Oncol 15(12):3588-600 (2008); Do T N, et al. Oncogene 22(10):1431-44 (2003); Kanovsky M, et al. Proc Natl Acad Sci USA 98(22):12438-43 (2001)). The limited value of cationic CPPs as a delivery vehicle for hydrophobic cargo underscores the need to develop alternative approaches for D-peptide activators of p53. The hydrocarbon stapling technique developed by Verdine and coworkers (Schafineister C E, et al. J Am Chem Soc 122: 5891-92 (2000); Bernal F, et al. J Am Chem Soc 129(9):2456-7 (2007); Moellering R E. Nature 462(7270):182-8 (2009); Walensky L D, et al. Science 305(5689):1466-70 (2004); Bernal F, et al. Cancer Cell 18(5):411-22 (2011)) represents a chemically robust, experimentally proven, and therapeutically viable approach to achieving highly efficient intracellular delivery of ^{D}PMI-δ. Hydrocarbon-stapled ^{D}PMI-δ should exert potent p53-dependent antitumor activity in vitro and in vivo, fulfilling the promise of D-peptide activators of p53 as a novel class of anticancer therapeutics.

Research Design.

A regular α-helix contains 3.6 residues per turn. Thus, the two residues at (i, i+4) positions are one turn apart, and two turns apart at (i, i+7) positions. This can be best illustrated by the left-handed helical structure of ^{D}PMI-α bound to MDM2 (FIG. 4). Each of Ala5, Lys9 and Arg12 can be selected for side-chain crosslinking—the three residues at (i, i+4, i+7) positions opposite the MDM2-binding interface as they are all non-contact residues and do not make any significant contribution to MDM2 binding (Liu M, et al. Proc Natl Acad Sci USA 107(32):14321-6 (2010); Liu M, et al. Angew Chem Int Ed Engl 49(21):3649-3652 (2010)).

Hydrocarbon Stapling.

Hydrocarbon-stapled ^{D}PMI-δ can be synthesized using the standard Fmoc chemistry, and crosslinking can be achieved on resin using Grubbs catalyst as described (Bernal F, et al. J Am Chem Soc 129(9):2456-7 (2007)). Three different types of geometries can be investigated, i.e., (i, i+4), (i+4, i+7), and (i, i+7). In addition, the stereochemistry (R or S) and length of the staples can be varied for optimization. AnaSpec is the exclusive provider of the non-classical amino acids bearing olefinic side chains for ruthenium-catalyzed olefin metathesis, including (R or S)—N-Fmoc-2-(2′-propenyl)alanine, (R or S)—N-Fmoc-2-(4′-pentenyl)alanine, and (R or S)—N-Fmoc-2-(7′-octenyl)alanine (Table 4). In a proof of principle study, PMI—an L-peptide antagonist of MDM2 and MDMX has been successfully stapled using (R)—N-Fmoc-2-(4′-pentenyl)alanine.

Functional and Structural Characterization.

Stapled peptides can be purified to homogeneity by RP-HPLC and ascertained by ESI-MS. CD spectroscopy can be used to determine α-helicity of the peptides both in aqueous solution and in the presence of trifluoroethanol. The binding affinity of the stapled ^{D}PMI-δ peptides for MDM2 and MDMX can be determined using our previously established competition SPR assay (Liu M, et al. Proc Natl Acad Sci USA 107(32):14321-6 (2010); Liu M, et al. Angew Chem Int Ed Engl 49(21):3649-3652 (2010); Pazgier M, et al. Proc Natl Acad Sci USA 106(12):4665-70 (2009); Li C, et al. Angew Chem Int Ed Engl 48(46):8712-5 (2009); Li C, et al. J Mol Biol 398(2):200-13 (2010)). In addition, fluorescence polarization techniques (Czarna A, et al. Cell Cycle 8(8):1176-84 (2009); Harker E A, et al. Bioorg Med Chem 17(5):2038-46 (2009); Heyduk T and Lee J C. Proc Natl Acad Sci USA 87(5):1744-8 (1990)) can be used to measure the ability of the stapled D-peptides to compete for MDM2 and MDMX binding with a fluorescently labeled p53 peptide—a task now routinely performed with a Tecan Infinite M1000 multimode microplate reader. Stapled ^{D}PMI-δ peptides with high binding affinity for MDM2 and/or MDMX can be selected for crystallographic studies. Diffraction data can be collected at the X-ray Crystallography Core Facility at the University of Maryland, Baltimore. The structures can be solved by molecular replacement using search models based on the previously determined and refined structures of MDMX/MDM2-peptide complexes including our own (Liu M, et al. Proc Natl Acad Sci USA 107(32):14321-6 (2010); Liu M, et al. Angew Chem Int Ed Engl 49(21):3649-3652 (2010); Pazgier M, et al. Proc Natl Acad Sci USA 106(12):4665-70 (2009); Li C, et al. Angew Chem Int Ed Engl 48(46):8712-5 (2009); Li C, et al. J Mol Biol 398(2):200-13 (2010)). To obtain higher resolution structures, crystals may be shipped to the Stanford Synchrotron Radiation Lightsource for data collection.

Cellular Uptake.

To evaluate uptake efficiency in HeLa cells using a four-channel Zeiss LSM 510 META laser-scanning microscope, selected stapled D-peptides can be N-terminally modified with fluorophores such as succinimidyl ester-activated BODIPY (Invitrogen). The cellular plasma membrane can be fluorescently labeled by wheat germ agglutinin (WGA)-conjugated Qdot 655 nanocrystals (Invitrogen) with high sensitivity and low non-specific binding. A quantitative fluorometric assay and HPLC-based cell lysate analysis protocol established by Holm T et al. may be use to quantify the amount of internalized D-peptides (Nat Protoc 1(2):1001-5 (2006)).

Free ^{D}PMI-δ is unstructured in aqueous solution and incapable of permeabilizing the cell membrane. Hydrocarbon stapling of _{D}PMI-δ can yield several D-peptides that adopt a helical conformation in buffer at room or lower temperature. Conformational stabilization will lead to enhanced ^{D}PMI-δ binding to MDM2 and MDMX due to a reduced energy loss in entropy. As was demonstrated with a stapled p53 peptide (Bernal F, et al. Cancer Cell 18(5):411-22 (2011)), hydrocarbon stapling of ^{D}PMI-δ may also preferentially improve MDMX binding, which can result in a dual-specificity D-peptide antagonist of exceptional potency. Structural studies of high-affinity D-peptides in complex with MDM2 and MDMX can show that the side-chain staple poses little or no structural perturbation to binding. Finally, it is expected that several hydrocarbon'-stapled ^{D}PMI-δ peptides with enhanced α-helicity and binding activity can be shown to efficiently traverse the cell membrane.

Weak in vitro activity could in principle stem from peptide entrapment in endosomes (Vives E, et al. Biochim Biophys Acta. 1786(2):126-38 (2008)). The N-terminal fusogenic peptide of the influenza virus hemagglutinin-2 subunit (HA2) induces lysis of membranes at low pHs. Co-administration of traces amounts of HA2 peptide can promote escape of endosome-trapped cargo into the cytosol (Wadia J S, et al. Nat Med 10(3):310-5 (2004); Yoshikawa T, et al. Mol Biol 380(5):777-82 (2008)), which has been confirmed in our laboratory with fluorescently labeled ^{D}PMI-δ (unpublished results). D-peptides may be co-administered with trace amounts of the HA2 peptide to augment uptake efficiency of hydrocarbon-stapled ^{D}PMI-δ peptides. Finally, additional mutations to increase cationicity may be needed to further improve membrane permeability of hydrocarbon-stapled peptides as indicated previously (Bernal F, et al. J Am Chem Soc 129(9):2456-7 (2007)).

### Example 5

### Testing the Hypothesis that High-Affinity, Protease-Resistant, and Cell-Penetrating D-Peptide Antagonists of MDM2 and MDMX Reactivate the p53 Pathway and Kill Tumor Cells through cell cycle arrest and/or apoptosis induction

Rationale.

p53 induces the expression of responsive genes involved in cell cycle arrest, senescence, or apoptosis. Treatment of wild type p53-harboring tumor cells with MDM2 and MDMX antagonists should, in principle, lead to (1) stabilization and accumulation of the p53 protein consequent to the blocking of its degradation; (2) activation of the p53 pathway as evidenced by p53-dependent induction of MDM2 expression; (3) activation of other p53-regulated genes such as p21 and PUMA responsible for cell cycle arrest and apoptosis, respectively. Analyses of these biological events can functionally and mechanistically validate D-peptide inhibitors of the p53-MDM2/MDMX interactions as a novel class of p53 activators with antitumor activity, paving the way for in vivo efficacy studies in animal models.

Research Design.

Stapled ^{D}PMI-δ peptides with enhanced activity and membrane permeability can be tested against a variety of appropriate cell lines. B. Vogelstein and colleagues constructed a panel of isogenic human colorectal cancer cell lines, SW48, DLD-1, RKO, and HCT116, differing only in their endogenous TP53 status (Sur S, et al. Proc Natl Acad Sci USA 106(10):3964-9 (2009)). For this study, we can use SW48, RKO and HCT116 cells that carry (1) two wild type alleles of TP53 (TP53^{+/+}), (2) one wild type TP53 allele and one disrupted allele (TP53^{+/-}), and (3) two disrupted TP53 alleles (TP53^{-/-}). DLD-1 cells normally have one allele that is mutant (S241F) and one allele that is not detectably expressed (TP53^{S241F/SIL}). For this study, we can use DLD-1 TP53^{S241F/SIL}, DLD-1 TP53^{-/SIL}, and DLD-1 TP53^{+/SIL}. We can also use Weril and Y79 human retinoblastoma cell lines (wild type p53) and the p53-deficient mouse retinoblastoma cell line SJMRBL-8 over-expressing MDMX (Laurie N A, et al. Nature 444(7115):61-6 (2006); Reed D, et al. J Biol Chem 285(14):10786-96 (2010)). Other commonly used cell lines to be tested that carry wild type p53 include but not limited to: SJSA-1 (osteosarcoma), MCF-7 (breast), LNCaP (prostate), and U87; cell lines with mutated/deleted p53 can be used as controls such as U251 and PC-3.

Specifically, we can first determine growth inhibitory activity of D-peptide antagonists against various cell lines using the standard MTS cell viability and trypan blue exclusion assays. Selected cell lines can be used for subsequent studies to elucidate the mechanisms of action of D-peptide antagonists in activating the p53 pathway. Western blotting and RT-PCR can be performed to analyze the expression of p53, MDM2, MDMX, p21, and PUMA in cells treated with different doses of D-peptide. Cell cycle and apoptosis can be analyzed by flow cytometry with propidium iodide and Annexin V staining. For verification, the caspase inhibitor Z-VAD-FMK can be used to block apoptosis induced by MDM2 and MDMX antagonists. Primary human and mouse fibroblasts (normal cells) and Nutlin-3 can be used as controls.

The following findings are expected. (1) D-peptide antagonists of MDM2 and MDMX potently inhibit, in a dose-dependent manner, the growth of tumor cells harboring wild type p53 but show little or no inhibitory activity against tumor cells with mutated or deleted p53. (2) In tumor cells harboring wild type p53, D-peptide antagonists of MDM2 and MDMX induce dose-dependent accumulation of the p53 protein (at the post-transcriptional level), expression of MDM2 and p21 or PUMA (at the transcriptional level), and MDM2-dependent degradation of MDMX. In isogenic tumor cell lines with one wild type TP53 allele and one disrupted allele (TP53^{+/-}), partial accumulation of p53 and expression of p53 responsive genes is expected. (3) Consequently, cell cycle arrest and apoptosis are observed in wild type p53-harboring tumor cells treated with D-peptide antagonists, but not in p53-null or p53 mutant tumor cells. (4) It has been reported that the MDM2 antagonist MI-219 causes transient activation of the p53 pathway in normal cells as evidenced by accumulation of p53 and increased expression of MDM2 and p21, but fails to induce the expression of PUMA and apoptosis (Shangary S, et al. Proc Natl Acad Sci USA 105(10):3933-8 (2008)). Thus, we expect to make similar findings on D-peptide antagonists of MDM2 and MDMX with respect to normal fibroblasts.

All documents, books, manuals, papers, patents, published patent applications, guides, abstracts and other reference materials cited herein, including GenBank Accession Numbers, are incorporated by reference in their entirety. While the foregoing specification teaches the principles of the present invention, with examples provided for the purpose of illustration, it will be appreciated by one skilled in the art from reading this disclosure that various changes in form and detail can be made without departing from the true scope of the invention.

What is claimed is:

1. A D-peptide variant that binds MDM2 or MDMX, or both, with high affinity and that is selected from the group consisting of the D-peptide variants set forth in SEQ ID NOs:3-34.

2. The D-peptide variant of claim 1, wherein the D-peptide variant is the D-peptide variant set forth in SEQ ID NO:13.

3. A pharmaceutical composition comprising a D-peptide variant of claim 1 and a pharmaceutical carrier and/or excipient.

4. The pharmaceutical composition of claim 3, wherein the D-peptide variant is set forth in SEQ ID NO:13.

5. A method of treating a subject having cancer, comprising administering to the subject having cancer a pharmaceutically-effective amount of a pharmaceutical composition of claim 3, thereby treating the subject having cancer.

6. The method of claim 5, wherein the D-peptide variant is set forth in SEQ ID NO:13.

7. The method of claim 5, wherein the subject is a human.

8. The method of claim 5, wherein the pharmaceutical carrier is a PEGylated liposome.

9. The method of claim 6, wherein the pharmaceutical carrier is a PEGylated liposome.

10. The method of claim 5, wherein the pharmaceutical carrier is a PEGylated liposome coated via a PEG spacer with a cyclic RGD peptide c(RGD^{D}YK).

11. The method of claim 6, wherein the pharmaceutical carrier is a PEGylated liposome coated via a PEG spacer with a cyclic RGD peptide c(RGD^{D}YK).

12. A composition comprising a D-peptide variant of claim 1 and a carrier and/or excipient.

13. The composition of claim 12, wherein the D-peptide variant is set forth in SEQ ID NO:13.

14. A composition comprising a D-peptide variant of claim 1 and a PEGylated liposome.

15. The composition of claim 14, wherein the D-peptide variant is set forth in SEQ ID NO:13.

16. The composition of claim 14 wherein the PEGylated liposome is coated via a PEG spacer with a cyclic RGD peptide c(RGD^{D}YK).

17. The composition of claim 15 wherein the PEGylated liposome is coated via a PEG spacer with a cyclic RGD peptide c(RGD^{D}YK).
